SEQUENCE LISTING (51 sequences)

SEQ ID NO: 1 - protein, 216 aa, Canis lupus
  source 1..216: note = Canis lupus familiaris
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 215 aa, Canis lupus
  source 1..215: note = Canis lupus familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 3 - protein, 215 aa, Canis lupus
  source 1..215: note = Canis lupus familiaris
LGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 4 - protein, 216 aa, Canis lupus
  source 1..216: note = Canis lupus familiaris
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 5 - protein, 445 aa, synthetic construct
  REGION 1..445: note = source = /note="Description of Artificial Sequence: Synthetic polypeptide"
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 6 - protein, 558 aa, synthetic construct
  REGION 1..558: note = source = /note="Description of Artificial Sequence: Synthetic polypeptide"
GGVAAPTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 7 - protein, 554 aa, synthetic construct
  REGION 1..554: note = source = /note="Description of Artificial Sequence: Synthetic polypeptide"
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 8 - protein, 445 aa, synthetic construct
  REGION 1..445: note = source = /note="Description of Artificial Sequence: Synthetic polypeptide"
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPPKRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 9 - protein, 558 aa, synthetic construct
  REGION 1..558: note = source = /note="Description of Artificial Sequence: Synthetic polypeptide"
GGVAAPTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 10 - protein, 554 aa, synthetic construct
  REGION 1..554: note = source = /note="Description of Artificial Sequence: Synthetic polypeptide"
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 11 - protein, 445 aa, synthetic construct
  REGION 1..445: note = source = /note="Description of Artificial Sequence: Synthetic polypeptide"
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 12 - protein, 445 aa, synthetic construct
  REGION 1..445: note = source = /note="Description of Artificial Sequence: Synthetic polypeptide"
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPPKRENGRVPRPPDCPKCPAPEMLGGPSVFIFPPKPKDTLYIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 13 - protein, 554 aa, synthetic construct
  REGION 1..554: note = source = /note="Description of Artificial Sequence: Synthetic polypeptide"
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLYIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 14 - protein, 451 aa, synthetic construct
  REGION 1..451: note = source = /note="Description of Artificial Sequence: Synthetic polypeptide"
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVWDLDPEDPEVQISWFVDGKQMQTAKTQPREEQFAGTYRWSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 15 - protein, 220 aa, synthetic construct
  REGION 1..220: note = source = /note="Description of Artificial Sequence: Synthetic polypeptide"
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIKRNDAQPAVYLFQPSPDQLHTGSASWCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 16 - protein, 452 aa, synthetic construct
  REGION 1..452: note = source = /note="Description of Artificial Sequence: Synthetic polypeptide"
EVQLVESGGDLVKPGGSLRLSCVASGFTFSNYGMSWVRQAPGKGLQWVATISYGGSYTYYPDNIKGRFTISRDNAKNTLYLQMNSLRAEDTAMYYCVRGYGYDTMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 17 - protein, 217 aa, synthetic construct
  REGION 1..217: note = source = /note="Description of Artificial Sequence: Synthetic polypeptide"
EIVMTQSPASLSLSQEEKVTITCKASQSVSFAGTGLMHWYQQKPGQAPKLLIYRASNLEAGVPSRFSGSGSGTDFSFTISSLEPEDVAVYYCQQSREYPWTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 18 - protein, 445 aa, synthetic construct
  REGION 1..445: note = source = /note="Description of Artificial Sequence: Synthetic polypeptide"
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVYPPSREELSKNTVSLTCLIKDFFPPDIDVEWQSNGQQEPESKYRTTPPQLDEDGSYALVSKLSVDKSRWQRGDTFICAVMHEALHNHYTQESLSHSPGK

SEQ ID NO: 19 - protein, 558 aa, synthetic construct
  REGION 1..558: note = source = /note="Description of Artificial Sequence: Synthetic polypeptide"
GGVAAPTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLLCLIKDFFPPDIDVEWQSNGQQEPESKYLTWPPQLDEDGSYFLYSKLSVDKSRWQRGDTFICAVMHEALHNHYTQESLSHSPGK

SEQ ID NO: 20 - protein, 554 aa, synthetic construct
  REGION 1..554: note = source = /note="Description of Artificial Sequence: Synthetic polypeptide"
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKETPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLLCLIKDFFPPDIDVEWQSNGQQEPESKYLTWPPQLDEDGSYFLYSKLSVDKSRWQRGDTFICAVMHEALHNHYTQESLSHSPGK

SEQ ID NO: 21 - protein, 17 aa, Canis lupus
  source 1..17: note = Canis lupus familiaris
FNECRCTDTPPCPVPEP

SEQ ID NO: 22 - protein, 22 aa, Canis lupus
  VARIANT 19: note = /replace="Ala"
  SITE 1..22: note = /note="Variant residues given in the sequence have nopreference with respect to those in the annotationsfor variant positions"
  source 1..22: note = Canis lupus familiaris
PKRENGRVPRPPDCPKCPTPEM

SEQ ID NO: 23 - protein, 20 aa, Canis lupus
  source 1..20: note = Canis lupus familiaris
AKECECKCNCNNCPCPGCGL

SEQ ID NO: 24 - protein, 17 aa, Canis lupus
  source 1..17: note = Canis lupus familiaris
PKESTCKCISPCPVPES

SEQ ID NO: 25 - protein, 452 aa, synthetic construct
  REGION 1..452: note = source = /note="Description of Artificial Sequence: Synthetic polypeptide"
EVQLVESGGGLVKPGRSMKLSCAASGFTFSNYYMAWVRQAPTKGLEWVASISTGGGNTYYRDSVKGRFTISRDNAKRTLYLQMDSLRSEDTATYYCGRHGTLYFDYWGQGVMVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 26 - protein, 452 aa, synthetic construct
  REGION 1..452: note = source = /note="Description of Artificial Sequence: Synthetic polypeptide"
EVQLVESGGDLVKPGGSLRLSCVASGFTFSNYYMAWVRQAPGKGLQWVASISTGGGNTYYRDSVKGRFTISRDNAKNTLYLQMNSLRAEDTAMYYCAKHGTLYFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 27 - protein, 452 aa, synthetic construct
  REGION 1..452: note = source = /note="Description of Artificial Sequence: Synthetic polypeptide"
EVQLVESGGDLVKPGGSLRLSCAASGFTFSNYYMAWVRQAPGKGLQWVASISTGGGNTYYRDSVKGRFTISRDNAKNTLYLQMNSLRAEDTAMYYCARHGTLYFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - protein, 218 aa, synthetic construct
  REGION 1..218: note = source = /note="Description of Artificial Sequence: Synthetic polypeptide"
QFTLTQPKSVSGSLRSTITIPCERSSGDIGDSYVSWYQQHLGRPPINVIYVDDQRPSEVSDRFSGSIDSSSNSASLTITDLQMDDEADYFCQSYDSNIDGPVFGGGTKLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 29 - protein, 218 aa, synthetic construct
  REGION 1..218: note = source = /note="Description of Artificial Sequence: Synthetic polypeptide"
QSVLTQPASVSGSLGQRVTISCERSSGDIGDSYVSWYQQLPGKAPSLLIYVDDQRPSGVPERFSGSKSGSSNSATLTITGLQAEDEADYYCQSYDSNIDGPVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 30 - protein, 218 aa, synthetic construct
  REGION 1..218: note = source = /note="Description of Artificial Sequence: Synthetic polypeptide"
QPVLTQPPSLSASLGTTARLTCERSSGDIGDSYVSWYQQKPGSPPRDLLYVDDQRPSGVSKSFSGSKDTSANAGLLLISGLQPEDEADYYCQSYDSNIDGPVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 31 - protein, 218 aa, synthetic construct
  REGION 1..218: note = source = /note="Description of Artificial Sequence: Synthetic polypeptide"
QPVLTQPPSLSASLGTTARLTCERSSGDIGDSYVSWYQQKPGSPPRDVIYVDDQRPSEVSKSFSGSKDTSANAGLLLISGLQPEDEADYFCQSYDSNIDGPVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 32 - protein, 457 aa, synthetic construct
  REGION 1..457: note = source = /note="Description of Artificial Sequence: Synthetic polypeptide"
EVQLVESDGGLAQPGRSLKLSCAASGFTFSDYYMAWVRQAPTKGLEWVATISYDGSSTYYRDSVRGRFTISRDNAKSTLYLQMDSLRSEDTATYYCARGPLTDWAPNWFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 33 - protein, 457 aa, synthetic construct
  REGION 1..457: note = source = /note="Description of Artificial Sequence: Synthetic polypeptide"
EVQLVESGGDLVKPGGSLRLSCVASGFTFSDYYMAWVRQAPGKGLQWVATISYDGSSTYYRDSVRGRFTISRDNAKNTLYLQMNSLRAEDTAMYYCAKGPLTDWAPNWFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 34 - protein, 457 aa, synthetic construct
  REGION 1..457: note = source = /note="Description of Artificial Sequence: Synthetic polypeptide"
EVQLVESGGDLVKPGGSLRLSCAASGFTFSDYYMAWVRQAPGKGLQWVATISYDGSSTYYRDSVRGRFTISRDNAKNTLYLQMNSLRAEDTAMYYCARGPLTDWAPNWFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 35 - protein, 217 aa, synthetic construct
  REGION 1..217: note = source = /note="Description of Artificial Sequence: Synthetic polypeptide"
DIQMTQSPASLSASLGETVTIQCQTSEDIYSGLAWYQQKPGKSPQFLIYGASRLEDGVPSRFSGSGSGTQYSLKISSMQTEDEGVYFCQQGLKYPNTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 36 - protein, 217 aa, synthetic construct
  REGION 1..217: note = source = /note="Description of Artificial Sequence: Synthetic polypeptide"
DIVMTQTPLSLSVSPGETASISCQTSEDIYSGLAWFRQKPGQSPQRLIYGASRLEDGVPDRFSGSGSGTDFTLRISTVEADDTGVYYCQQGLKYPNTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 37 - protein, 217 aa, synthetic construct
  REGION 1..217: note = source = /note="Description of Artificial Sequence: Synthetic polypeptide"
EIVMTQSPASLSLSQEEKVTITCQTSEDIYSGLAWYQQKPGQAPKLLIYGASRLEDGVPSRFSGSGSGTDFSFTISSLEPEDVAVYYCQQGLKYPNTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 38 - protein, 217 aa, synthetic construct
  REGION 1..217: note = source = /note="Description of Artificial Sequence: Synthetic polypeptide"
DIVMTQSPASLSLSQEEKVTITCQTSEDIYSGLAWYQQKPGQAPKLLIYGASRLEDGVPSRFSGSGSGTDFSFTISSLEPEDVAVYFCQQGLKYPNTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 39 - protein, 217 aa, synthetic construct
  REGION 1..217: note = source = /note="Description of Artificial Sequence: Synthetic polypeptide"
DIVMTQTPLSLSVSPGETASISCQTSEDIYSGLAWFRQKPGQSPQLLIYGASRLEDGVPDRFSGSGSGTDFTLRISTVEADDTGVYFCQQGLKYPNTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 40 - protein, 455 aa, synthetic construct
  REGION 1..455: note = source = /note="Description of Artificial Sequence: Synthetic polypeptide"
QVQLKESGPGLVQPSQTLSLTCTVSGFSLTSNGVSWVRQPPGKGLEWIAAISSGGSTYYNSVLKSRLSISRDTSKSQVFLKMNSLQTEDTAIYFCTRRLSGYNYVPFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 41 - protein, 455 aa, synthetic construct
  REGION 1..455: note = source = /note="Description of Artificial Sequence: Synthetic polypeptide"
EVQLVESGGDLVKPEGSLRLSCVVSGFTFSSNGVSWVRQAPGKGLQWVAAISSGGSTYYNSVLKSRFTISRDNAKNTLYLQMNSLRTEDTAVYYCAKRLSGYNYVPFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 42 - protein, 455 aa, synthetic construct
  REGION 1..455: note = source = /note="Description of Artificial Sequence: Synthetic polypeptide"
ELTLQESGPGLVKPSQTLSLTCVVSGGSVTSNGVSWIRQRPGRGLEWMGAISSGGSTYYNSVLKSRISITADTAKNQFSLQLSSMTTEDTAVYYCARRLSGYNYVPFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - protein, 455 aa, synthetic construct
  REGION 1..455: note = source = /note="Description of Artificial Sequence: Synthetic polypeptide"
ELTLQESGPGLVKPSQTLSLTCTVSGFSLTSNGVSWIRQRPGRGLEWMGAISSGGSTYYNSVLKSRISITADTAKNQFSLQLSSMTTEDTAVYYCARRLSGYNYVPFAYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 44 - protein, 216 aa, synthetic construct
  REGION 1..216: note = source = /note="Description of Artificial Sequence: Synthetic polypeptide"
DIQMTQSPSLLSASVGDRVTLNCKASQNIYKHLAWCQQKLGEPPNLLISNANSLQTGIPSRFSGSGSGTDFTLTISSLQPEDVATYFCQQYYSGDTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 45 - protein, 216 aa, synthetic construct
  REGION 1..216: note = source = /note="Description of Artificial Sequence: Synthetic polypeptide"
EIVMTQSPASLSLSQEEKVTITCKASQNIYKHLAWYQQKPGQAPKLLIYNANSLQTGVPSRFSGSGSGTDFSFTISSLEPEDVAVYYCQQYYSGDTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 46 - protein, 216 aa, synthetic construct
  REGION 1..216: note = source = /note="Description of Artificial Sequence: Synthetic polypeptide"
EIVMTQSPASLSLSQEEKVTITCKASQNIYKHLAWYQQKPGQAPKLLIYNANSLQTGIPSRFSGSGSGTDFSFTISSLEPEDVAVYFCQQYYSGDTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 47 - protein, 216 aa, synthetic construct
  REGION 1..216: note = source = /note="Description of Artificial Sequence: Synthetic polypeptide"
EIVMTQSPGSLAGSAGESVSINCKASQNIYKHLAWYQQKPGERPKLLIYNANSLQTGVPARFSSSGSGTDFTLTINNLQAEDVGDYYCQQYYSGDTFGAGTKVELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 48 - protein, 208 aa, Canis lupus
  source 1..208: note = Canis lupus familiaris
VKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLP

SEQ ID NO: 49 - protein, 321 aa, Canis lupus
  source 1..321: note = Canis lupus familiaris
GGVAAPTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPT

SEQ ID NO: 50 - protein, 317 aa, Canis lupus
  source 1..317: note = Canis lupus familiaris
SMLSNAEIKVNPPQDFEIVDPGYLGYLSLQWQPPLFPDNFKECTIEYELKYRNIDSENWKTIITKNLHYKDGFDLNKGIEAKINTLLPAQCTNGSEVRSSWAETTYWTSPQGNRETKIQDMDCVYYNWQYLVCSWKPGMGVHFDTNYQLFYWYEGLDHSAECTDYIKVNGKNMGCRFPYLESSDYKDFYICVNGSSESQPIRPSYFIFQLQNIVKPMPPDYLSLTVKNSEEINLKWNMPKGPIPAKCFIYEIEFTEDGTTWVTTTVENEIQITRTSNESQKLCFLVRSKVNIYCSDDGIWSEWSDEQCWKGDIWKET

SEQ ID NO: 51 - protein, 215 aa, Canis lupus
  source 1..215: note = Canis lupus familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

(19) United States
(12) Patent Application Publication
(10) Pub. No.: US 2025/0325630 A1
(43) Pub. Date: Oct. 23, 2025

(54) HOMODIMER FUSION PROTEINS FOR TREATING ATOPIC DERMATITIS

(71) Applicant: Intervet Inc., Rahway, NJ (US)

(72) Inventors: Mohamad Morsey, Omaha, NE (US); Yuanzheng Zhang, Somerset, NJ (US); Seth D. Stauffer, Elkhorn, NE (US); Zhisong Qiao, Omaha, NE (US)

(73) Assignee: Intervet Inc., Rahway, NJ (US)

(21) Appl. No.: 18/684,254

(22) PCT Filed: Aug. 19, 2022
(86) PCT No.: PCT/EP2022/073147

§ 371 Date: Feb. 16, 2024

Related U.S. Application Data
(60) Provisional application No. 63/235,259, filed on Aug. 20, 2021.
(60) Provisional application No. 63/235,261, filed on Aug. 20, 2021.

(51) Int. Cl. A61K 38/17; A61K 31/4439; A61K 31/519; A61K 39/00; A61K 39/395

(52) U.S. Cl. CPC A61K 38/1793; A61K 31/4439; A61K 31/519; A61K 39/3955; A61K 2039/505

(57) ABSTRACT

The present invention provides compositions of homodimers of canine Interleukin-4 receptor alpha fusion proteins and canine Interleukin-13 receptor alpha 2 fusion proteins for treating canine atopic dermatitis. The compositions can further comprise a caninized antibody against canine IL-31 or a caninized antibody against canine IL-31Rα.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14463-292-999_SUB_SEQ_LISTING.xml”, was created on Sep. 12, 2024, and is 81,969 bytes in size.

### CROSS-REFERENCE TO RELATED APPLICATIONS

This application is a national stage application of International Patent Application No. PCT/EP2022/073147 filed Aug. 19, 2022, which claims priority under 35 U.S.C. § 119 (e) of U.S. Provisional Patent Application No. 63/235,259, filed Aug. 20, 2021 and U.S. Provisional Patent Application No. 63/235,261, filed Aug. 20, 2021, each of which is incorporated by reference in its entirety herein.

### FIELD OF THE INVENTION

The present invention relates to compositions for treating atopic dermatitis in canines that comprise fusion proteins that bind to canine interleukin-4 or canine interleukin-13. The compositions can be used to treat canine atopic dermatitis.

### BACKGROUND OF THE INVENTION

The immune system comprises a network of resident and recirculating specialized cells that function collaboratively to protect the host against infectious diseases and cancer. The ability of the immune system to perform this function depends to a large extent on the biological activities of a group of proteins secreted by leukocytes and collectively referred to as interleukins. Among the well-studied interleukins are three important molecules identified as: interleukin-4 (IL-4), interleukin-13 (IL-13), and interleukin-31 (IL-31). IL-4 and IL-13 are critical cytokines in related signaling pathways involved in the development of immune responses that are required for protection against certain pathogens (e.g., tissue or lumen dwelling parasites). However, these two cytokines, along with IL-31 also have been implicated in the pathogenesis of allergic diseases in humans and animals, including atopic dermatitis.

Atopic dermatitis (AD) is a relapsing pruritic and chronic inflammatory skin disease, that is characterized by immune system dysregulation and epidermal barrier abnormalities in humans. The pathological and immunological attributes of atopic dermatitis have been the subject of extensive investigations [reviewed in Rahman et al. Inflammation & Allergy-drug target 10:486-496 (2011) and Harskamp et al., Seminar in Cutaneous Medicine and Surgery 32:132-139 (2013)]. Atopic dermatitis also is a common condition in companion animals, especially dogs, where its prevalence has been estimated to be approximately 10-15% of the canine population. The pathogenesis of atopic dermatitis in dogs and cats [reviewed in Nuttall et al., Veterinary Records 172 (8): 201-207 (2013)] shows significant similarities to that of atopic dermatitis in man including skin infiltration by a variety of immune cells and CD4+Th2 polarized cytokine milicu including the preponderance of IL-4, IL-13, and IL-31.

IL-4 and IL-13 are closely related proteins that can be secreted by many cell types including CD4+Th2 cells, natural killer T cells (NKT), macrophages, mast cells, and basophils. IL-4 and IL-13 display many overlapping functions and are critical to the development of T cell-dependent humoral immune responses. Both IL-4 and IL-13 are part of a signaling pathway involved in atopic dermatitis. IL-4 binds to a heterodimeric receptor, which comprises a monomer of the common γc chain (γc) and a monomer of the IL-4 receptor alpha (IL-4Rα) respectively, whereas IL-13 binds to a heterodimeric receptor comprising a monomer of the IL-13 receptor alpha 1 (IL13Rα1) and a monomer of the IL-4Rα respectively.

Accordingly, the Th2 cytokines IL-4, IL-13, and IL-31 have been the object of therapeutic intervention in order to develop better therapies. Pharmaceuticals that have either proven to aid in the treatment of atopic dermatitis and/or have shown promise to do so include: Janus kinase (JAK) inhibitors [see e.g., U.S. Pat. Nos. 8,133,899; 8,987,283; WO 2018/108969; US 2020/0339585], spleen tyrosine kinase (SYK) inhibitors [see e.g., U.S. Pat. No. 8,759,366], and antagonists to a chemoattractant receptor-homologous molecule expressed on TH2 cells [see e.g., U.S. Pat. Nos. 7,696,222, 8,546,422, 8,637,541, and 8,546,422]. In addition, US 2020/0048325 A1 discloses contiguous IL-13/IL-4 receptor fusion proteins. The design of these fusion proteins brings together the IL-13Rα1 and IL-4Rα in a contiguous arrangement wherein the IL-13Rα1 is linked to the IL-4Rα by a non-self amino acid sequence called a linker and the contiguous receptors also may be linked to a fusion partner with a second non-self amino acid linker. Notably, the linkers used also have the potential to undergo post-translational modifications, e.g., glycosylation.

The therapeutic use of monoclonal antibodies to block signal transduction in specific pathways by binding to either a protein ligand or its protein receptor has proven to be widely successful. Indeed, such monoclonal antibodies play a critical role in the rapid growth of human biopharmaceuticals and as of 2017, claimed over 25% of the human biopharmaceutical market. Among the 20 drugs with the highest sales in 2014, six were monoclonal antibodies [Chung, Experimental & Molecular Medicine 49: e304; doi: 10.1038/emm.2017.46 (2017)]. This trend continues to grow. Monoclonal antibodies raised against human IL-4 receptor alpha (IL-4 Rα) have been developed and some of these antibodies have been extensively tested for their therapeutic effects for treating atopic dermatitis in humans [see, e.g., US2015/0017176 A1]. One such antibody (dupilumab) was produced by the immunization of transgenic mice in which the mouse antibody genes were replaced with human antibody genes and therefore, the resulting antibody is a human antibody as opposed to e.g., a humanized murine antibody.

Although initially limited to human biopharmaceuticals because of the high cost of monoclonal antibody therapeutics, canine monoclonal products have recently become available due to significant reductions in production costs. Early indications suggest that such monoclonal antibodies also are likely to become major therapeutics in the companion animal market as well. For example, an antibody against human IL-31 receptor alpha (IL-31RA) has been tested and found to have a significant effect on pruritus associated with atopic dermatitis in humans [Ruzicka, et al., New England Journal of Medicine, 376 (9), 826-835 (2017)]. In addition, antibodies against canine IL-31 have been shown to have a significant effect on pruritus associated with atopic dermatitis in dogs [U.S. Pat. No. 8,790,651 B2; U.S. Pat. No. 10,093,731 B2]. This caninized antibody blocks the binding of cIL-31 to the canine IL-31 receptor (cIL-31R), thereby blocking the cIL-31/cIL-31R signaling pathway. Accordingly, blocking IL-31 binding to its receptor IL-31RA, results in the relief of pruritus associated with atopic dermatitis. However, merely blocking the cIL-31/cIL-31R signaling pathway only ameliorates the pruritic effect of atopic dermatitis, but does nothing to stop the concomitant skin inflammation caused by the canine IL-4 (cIL-4) or canine IL-13 (cIL-13)/canine IL-4 receptor alpha (cIL-4Rα) signaling pathways. More recently, caninized antibodies to canine IL-4Rα that block the binding of canine IL-4 to canine IL-4Rα also have been disclosed [US2018/0346580A1, hereby incorporated by reference in its entirety]. These antibodies were produced by immunization of conventional, i.e., non-transgenic mice, with the canine IL-4Rα extra-cellular domain (ECD). Because the Type II IL-4 receptor consists of the IL-4Rα chain and the IL-13R al chain, antibodies to canine IL-4 Rα have been obtained that can block both canine IL-4 and canine IL-13 from binding the Type II canine IL-4 receptor, thereby serving to help block the inflammation associated with atopic dermatitis.

However, despite recent successes in treating pruritus associated with atopic dermatitis, and recent encouraging disclosures on the treatment of the associated inflammation, many subjects suffering from this condition still do not experience a rapid onset of antipruritic action concomitant with a significant effect on the skin inflammation. Therefore, there is a need to design alternative therapies to address this unmet medical need.

The citation of any reference herein should not be construed as an admission that such reference is available as “prior art” to the instant application.

### SUMMARY OF THE INVENTION

The present invention provides compositions that can be used to treat atopic dermatitis. The compositions can comprise fusion proteins that bind canine IL-4 along with fusion proteins that bind canine IL-13. In particular embodiments, the composition comprises a homodimer that comprises a pair of canine Interleukin-4 receptor alpha-canine fragment crystallizable region fusion proteins (cIL-4Rα-cFc fusion proteins) and a homodimer comprising a pair of canine Interleukin-13 receptor alpha 2-canine fragment crystallizable region fusion proteins (cIL-13Rα2-cFc fusion proteins), in which each of the pair of the cIL-4Rα-cFc fusion proteins comprises an extracellular domain (ECD) of canine Interleukin-4 receptor alpha (cIL-4Rα) or fragment thereof that binds canine Interleukin-4 (cIL-4), and a cFc (denoted herein as the first cFc), and each of the pair of the cIL-13Rα2-cFc fusion proteins comprises an extracellular domain (ECD) of canine Interleukin-13 receptor alpha 2 (cIL-13Rα2) or fragment thereof that binds canine Interleukin-13 (cIL-13), and a cFc (denoted herein as the second cFc). In certain embodiments the first cFc and the second cFc are the same. In other embodiments, the first cFc and the second cFc are different.

In particular embodiments of the compositions, the first cFc comprises an amino acid sequence that has at least 90%, 95%, 99%, or 100% identity with the amino acid sequence of SEQ ID NO: 1. In other embodiments, the first cFc comprises an amino acid sequence that has at least 90%, 95%, 99%, or 100% identity with the amino acid sequence of SEQ ID NO: 2. In still other embodiments, the first cFc comprises an amino acid sequence that has at least 90%, 95%, 99%, or 100% identity with the amino acid sequence of SEQ ID NO: 51. In yet other embodiments, the first cFc comprises an amino acid sequence that has at least 90%, 95%, 99%, or 100% identity with the amino acid sequence of SEQ ID NO: 3. In still other embodiments, the first cFc comprises an amino acid sequence that has at least 90%, 95%, 99%, or 100% identity with the amino acid sequence of SEQ ID NO: 4.

In certain embodiments of the compositions, the second cFc comprises an amino acid sequence that has at least 90%, 95%, 99%, or 100% identity with the amino acid sequence of SEQ ID NO: 1. In other embodiments, the second cFc comprises an amino acid sequence that has at least 90%, 95%, 99%, or 100% identity with the amino acid sequence of SEQ ID NO: 2. In still other embodiments, the second cFc comprises an amino acid sequence that has at least 90%, 95%, 99%, or 100% identity with the amino acid sequence of SEQ ID NO: 51. In yet other embodiments, the second cFc comprises an amino acid sequence that has at least 90%, 95%, 99%, or 100% identity with the amino acid sequence of SEQ ID NO: 3. In still other embodiments, the second cFc comprises an amino acid sequence that has at least 90%, 95%, 99%, or 100% identity with the amino acid sequence of SEQ ID NO: 4. In particular embodiments, the first cFc and the second cFc are the same. In other embodiments, the first cFc and the second cFc are different.

In certain embodiments of the compositions, the cIL-4Rα-cFc fusion protein further comprises a canine hinge region (denoted herein as the first canine hinge region). In related embodiments, the cIL-13Rα2-cFc fusion protein further comprises a canine hinge region (denoted herein as the second canine hinge region). In particular embodiments, the first canine hinge region and the second canine hinge region are the same. In other embodiments, the first canine hinge region and the second canine hinge region are different. A canine hinge region can act as a linker between the ECD of the cIL-4Rα and the first cFc and as a linker between the ECD of the cIL-13Rα2 and the second cFc.

In particular embodiments of the compositions, the first canine hinge region comprises an amino acid sequence that has at least 85%, 90%, 95%, or 100% identity with the amino acid sequence of SEQ ID NO: 21. In other embodiments, the first canine hinge region comprises an amino acid sequence that has at least 85%, 90%, 95%, or 100% identity with the amino acid sequence of SEQ ID NO: 22. In yet other embodiments, the first canine hinge region comprises an amino acid sequence that has at least 85%, 90%, 95%, or 100% identity with the amino acid sequence of SEQ ID NO: 23. In still other embodiments, the first canine hinge region comprises an amino acid sequence that has at least 85%, 90%, 95%, or 100% identity with the amino acid sequence of SEQ ID NO: 24.

In certain embodiments of the compositions, the second canine hinge region comprises an amino acid sequence that has at least 85%, 90%, 95%, or 100% identity with the amino acid sequence of SEQ ID NO: 21. In other embodiments, the second canine hinge region comprises an amino acid sequence that has at least 85%, 90%, 95%, or 100% identity with the amino acid sequence of SEQ ID NO: 22. In yet other embodiments, the second canine hinge region comprises an amino acid sequence that has at least 85%, 90%, 95%, or 100% identity with the amino acid sequence of SEQ ID NO: 23. In still other embodiments, the first canine hinge region comprises an amino acid sequence that has at least 85%, 90%, 95%, or 100% identity with the amino acid sequence of SEQ ID NO: 24. In particular embodiments, the first canine hinge region and the second canine hinge region are the same. In other embodiments, the first canine hinge region and the second canine hinge region are different.

In particular embodiments the canine hinge region and the cFc are both from IgGA. In other embodiments the canine hinge region and the cFc are both from IgGB. In still other embodiments the canine hinge region and the cFc are both from IgGC. In yet other embodiments the canine hinge region and the cFc are both from IgGD.

In certain embodiments of the compositions, the ECD of cIL-4Rα comprises at least 85%, 90%, 95%, or 100% identity with the amino acid sequence of SEQ ID NO: 48. In other embodiments the ECD of cIL-13Rα2 comprises at least 85%, 90%, 95%, or 100% identity with the amino acid sequence of SEQ ID NO: 50. In still other embodiments the ECD of cIL-4Rα comprises at least 85%, 90%, 95%, or 100% identity with the amino acid sequence of SEQ ID NO: 48 and the ECD of cIL-13Rα2 comprises at least 85%, 90%, 95%, or 100% identity with the amino acid sequence of SEQ ID NO: 50.

In specific embodiments of the compositions, the sole linker between the ECD of the cIL-4Rα and the first cFc comprises an amino acid sequence that is identical to an amino acid sequence in a protein naturally found in canines, including a naturally occurring variant thereof. In related embodiments, the first canine hinge region acts as the sole linker between the ECD of the cIL-4Rα and the first cFc. In other specific embodiments, the sole linker between the ECD of the cIL-13Rα2 and the second cFc comprises an amino acid sequence that is identical to an amino acid sequence in a protein naturally found in canines, including a naturally occurring variant thereof. In related embodiments, the second canine hinge region acts as the sole linker between the ECD of the cIL-13Rα2 and the second cFc.

In more specific embodiments of the compositions, the sole linker between the ECD of the cIL-4Rα and the first cFc comprises an amino acid sequence that is identical to an amino acid sequence in a protein naturally found in canines, including a naturally occurring variant thereof and the sole linker between the ECD of the cIL-13Rα2 and the second cFc comprises an amino acid sequence that is identical to an amino acid sequence in a protein naturally found in canines including a naturally occurring variant thereof. In related embodiments, the first canine hinge region acts as the sole linker between the ECD of the cIL-4Rα and the first cFc, and the second canine hinge region acts as the sole linker between the ECD of the cIL-13Rα2 and the second cFc.

In particular embodiments of the compositions, the cIL-4Rα-cFc fusion protein is composed solely of amino acid sequences that are identical to amino acids sequences of proteins naturally found in canines, including naturally occurring variants thereof. In related embodiments, the cIL-13Rα2-cFc fusion protein is composed solely of amino acid sequences that are identical to amino acids sequences of proteins naturally found in canines, including naturally occurring variants thereof. In specific embodiments, both the cIL-4Rα-cFc fusion protein and the cIL-13Rα2-cFc fusion protein is composed solely of amino acid sequences naturally found in canines, including naturally occurring variants thereof.

In certain embodiments of the compositions, the cIL-4Rα-cFc fusion protein comprises an amino acid sequence that has at least 90%, 95%, or 99% identity with the amino acid sequence of SEQ ID NO: 5. In particular embodiments, the cIL-4Rα-cFc fusion protein comprises the amino acid sequence of SEQ ID NO: 5. In other embodiments, the cIL-4Rα-cFc fusion protein comprises the amino acid sequence of SEQ ID NO: 8. In still other embodiments, the cIL-4Rα-cFc fusion protein comprises the amino acid sequence of SEQ ID NO: 11. In yet other embodiments, the cIL-4Rα-cFc fusion protein comprises the amino acid sequence of SEQ ID NO: 12.

In specific embodiments of the compositions, the cIL-13Rα2-cFc fusion protein comprises an amino acid sequence that has at least 90%, 95%, or 99% identity with the amino acid sequence of SEQ ID NO: 7. In particular embodiments, the cIL-13Rα2-cFc fusion protein comprises the amino acid sequence of SEQ ID NO: 7. In other embodiments, the cIL-13Rα2-cFc fusion protein comprises the amino acid sequence of SEQ ID NO: 10. In still other embodiments, the cIL-13Rα2-cFc fusion protein comprises the amino acid sequence of SEQ ID NO: 13.

Any of the compositions of the present invention can further comprise an antipruritic antibody. In particular embodiments, the antipruritic antibody is a canine antibody. In more particular embodiments, the antipruritic antibody is a canine antibody against canine Interleukin-31 (cIL-31). In other embodiments, the antipruritic antibody is a caninized antibody. In particular embodiments, the caninized anti-pruritic antibody is an antibody against cIL-31. In more particular embodiments, the caninized antibody against cIL-31 comprises a heavy chain comprising the amino acid sequence of SEQ ID NO: 14 and a light chain comprising the amino acid sequence of SEQ ID NO: 15. In alternative embodiments, the caninized antibody against cIL-31 comprises a heavy chain comprising the amino acid sequence of SEQ ID NO: 16 and a light chain comprising the amino acid sequence of SEQ ID NO: 17.

In other embodiments of the compositions, the antipruritic antibody is a canine antibody against the canine Interleukin-31R (cIL-31R). In certain embodiments, the antipruritic antibody is a caninized antibody against cIL-31R. In yet other embodiments, the caninized antibody against cIL-31R comprises a heavy chain comprising the amino acid sequence of SEQ ID NO: 26 or SEQ ID NO: 27 and a light chain comprising the amino acid sequence of SEQ ID NO: 29, SEQ ID NO: 30, or SEQ ID NO: 31. In still other embodiments, the caninized antibody against cIL-31R comprises a heavy chain comprising the amino acid sequence of SEQ ID NO: 33 or SEQ ID NO: 34 and a light chain comprising the amino acid sequence of SEQ ID NO: 36, SEQ ID NO: 37, SEQ ID NO: 38, or SEQ ID NO: 39. In yet other embodiments, the caninized antibody against cIL-31R comprises a heavy chain comprising the amino acid sequence of SEQ ID NO: 41, SEQ ID NO: 42, or SEQ ID NO: 43 and a light chain comprising the amino acid sequence of SEQ ID NO: 45, SEQ ID NO: 46, or SEQ ID NO: 47.

Any of the compositions of the present invention also can further comprise one or more additional therapeutic components. In particular embodiments, the additional therapeutic component is a Janus kinase (JAK) inhibitor. In other embodiments, the additional therapeutic component is a spleen tyrosine kinase (SYK) inhibitor. In still other embodiments, the additional therapeutic component is an antagonist to a chemoattractant receptor-homologous molecule expressed on TH2 cells.

In specific embodiments, the JAK inhibitor is:

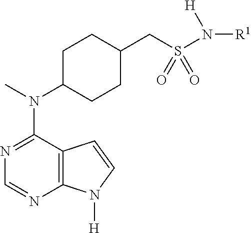

where R¹ is C₁₋₄ alkyl optionally substituted with hydroxy, and pharmaceutically acceptable salts thereof.

In alternative embodiments, the JAK inhibitor is:

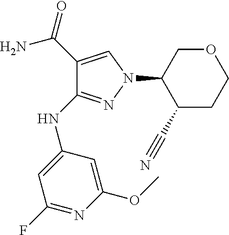 [SMILES: COc1cc(Nc2nn([C@H]3COCC[C@@H]3C#N)cc2C(N)=O)cc(F)n1]

and pharmaceutically acceptable salts thereof.

In yet other embodiments, the JAK inhibitor is:

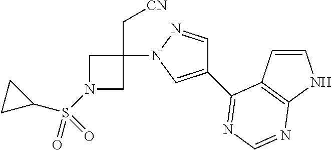 [SMILES: N#CCC1(n2cc(-c3ncnc4[nH]ccc34)cn2)CN(S(=O)(=O)C2CC2)C1]

and pharmaceutically acceptable salts thereof.

The present invention further includes method of treating atopic dermatitis comprising administering any of the compositions of the present invention to a canine that has atopic dermatitis.

These and other aspects of the present invention will be better appreciated by reference to the following Brief Description of the Drawings and the Detailed Description.

### BRIEF DESCRIPTION OF THE DRAWINGS

FIG. 1 depicts the binding activity of chimeric and caninized anti-canine IL-31Rα antibodies as evaluated by ELISA. Chimeric rat/canine 44E2 [●]. Caninized 44E2: H2k1 [ [symbol] ], H2k2 [▴], H5k1 [▾], and H5k2 [♦].

FIG. 2 depicts the binding activity of chimeric and caninized anti-canine IL-31Rα antibodies evaluated by ELISA. Chimeric rat/canine: 10A12 [●]. Caninized 10A12: H1L5 [ [symbol] ] and H2L6 [▴].

FIG. 3 depicts the binding activity of chimeric and corresponding caninized anti-canine IL-31Rα antibodies evaluated by ELISA. Chimeric rat/canine 28F12. Caninized 28F12: H1k3 [ [symbol] ], H2k2 [▴], and H2k3 [▾].

### DETAILED DESCRIPTION OF THE INVENTION

The successful therapeutic use of monoclonal antibodies to block signal transduction in specific pathways by binding to either a protein ligand or its protein receptor mentioned above, was an impetus to generate caninized antibodies against canine IL-4 receptor alpha. Accordingly, murine antibodies were raised against cIL-4Rα, then caninized, and shown in vitro, to effectively block the binding of cIL-4Rα with either of its two natural ligands, i.e., cIL-4 or cIL-13. Surprisingly however, unusually high amounts of so-called anti-drug antibodies (ADA) were detected in the treated canines after the caninized murine cIL-4Rα antibodies were administered to dogs. Even more unexpectedly, this issue arose for multiple different caninized murine cIL-4Rα antibodies that were tested.

The induction of ADA is a substantial obstacle in the development of monoclonal antibodies as therapeutics. ADAs are antibodies formed by the animal subject against the therapeutic antibody (i.e. the drug) that is administered to the animal subject. They typically neutralize the biological activity of the therapeutic antibody and/or lead to rapid clearance of the therapeutic antibody from the systemic circulation of the animal subject to which they are administered. The problem of ADA becomes more severe when the antibodies are initially generated in one species e.g., mice or rats, but are used to make a therapeutic antibody for a second species, e.g., canines, which is the way caninized murine or rat antibodies are constructed.

Moreover, in order to retain the strong binding affinity of the selected rat antibody for the target canine protein in the corresponding caninized rat antibody, it is generally necessary to include not only the amino acid sequences of the mouse or rat CDRs, but to also include additional amino acid residues from the amino acid sequence of the mouse or rat antibody. These additional amino acids are termed back mutations. The back mutations serve to maintain the three-dimensional structure of the CDRs and thereby facilitate the retention of the strong binding affinity of the mouse or rat antibody for the canine target protein in the caninized mouse or rat antibody. However, increasing the number of mouse or rat amino acid residues into the therapeutic caninized mouse or rat antibody, i.e., through the addition of the mouse or rat CDRs and the related back mutations, also increases the likelihood of that antibody being recognized as foreign by the immune system of the dog being treated, which results in ADA.

As indicated above, whereas the occurrence of ADA is a common issue for most therapeutic antibodies, it generally is regarded as a manageable problem because it normally occurs in a relatively small sub-population of those being treated. Surprisingly however, the number of dogs treated with the caninized murine cIL-4Rα antibodies that exhibited ADA proved to be unexpectedly high. Without limiting the explanation for this surprising result to any specific molecular mechanism, in retrospect, the fact that cIL-4Rα is expressed on antigen presenting cells (APC) may be an important factor. Accordingly, the binding of the therapeutic caninized cIL-4Rα antibodies to the cIL-4Rα of the APC could lead to the internalization of the bound cIL-4Rα. This would be followed by the subsequent presentation of protein fragments having sequences containing the murine CDRs (or the murine CDRs and the murine back mutations) of the caninized antibody to canine T cells, which could lead to the observed higher induction of ADA in the treated animals.

Regardless of the cause of the elevated number of dogs treated with caninized murine cIL-4Rα antibodies exhibiting ADA, its discovery led to the evaluation of alternative strategies for blocking the cIL-4 or cIL-13/cIL-4Rα signaling pathway. One potential alternative strategy is to directly block cIL-4 and cIL-13, rather than cIL-4Rα which, as noted above, is a part of both the canine IL-4 receptor and the canine IL-13 receptor. A possible methodology to accomplish this goal would be through the use of the extracellular domains (ECD) of two naturally occurring binding partners of IL-4 and IL-13, i.e., the ECD of IL-4Rα and the ECD of IL-13Rα1, respectively.

A currently popular methodology that could be employed would be the use of a contiguous bispecific fusion protein comprising both the ECD of IL-4Rα and ECD of IL-13Rα1. Contiguous bispecific fusion proteins have definite advantages, such as allowing the synthesis of a single therapeutic protein molecule rather than requiring synthesizing two separate protein molecules. In addition, if the two functional components of the bispecific fusion protein are functionally related, as in the case of a contiguous bispecific cIL-13Rα1 and cIL-4Rα fusion protein, a synergy would be expected because the binding of the first functional component (e.g., cIL-13Rα1) would be expected to facilitate the binding of the second functional component (e.g., cIL-4Rα). One such strategy has been proffered, which employs contiguous IL-13/IL-4 receptor fusion proteins [see, US2020/0048325 A1]. The design of such fusion proteins brings together the IL-13Rα1 and IL-4Rα in a contiguous arrangement in which the IL-13Rα1 is linked to the IL-4Rα. In addition, the contiguous receptors also may be linked to a fusion partner with a second linker. However, a significant disadvantage of such methodology is the use of such linkers, which are generally unnatural constituents of the fused receptors, and thereby could lead to potential neoepitopes that could induce ADA formation. In addition, the linkers used further have the potential to undergo post-translational modifications (e.g., glycosylation), which could create variant molecules with potentially altered structure that, in turn, could further lead to ADA formation.

An alternative method for creating a bispecific fusion protein is the use of bispecific heterodimers of fusion proteins of the ECD of IL-13Rα1 and the ECD of IL-4Rα [WO2020/086886] or the ECD of IL-13Rα2 and the ECD of IL-4Rα. Yet another putative strategy is the use of canine Fc fusion proteins incorporating homodimers of IL-4Rα-cFc fusion proteins combined with homodimers of IL-13Rα1-cFc fusion proteins and/or IL-13Rα2-cFc fusion proteins. In either case, these ECD's can be fused with a canine IgG (cFc), i.e., IgGA, IgGB, IgGC, or IgGD. More preferably, the fusion proteins can comprise a canine IgG hinge region or fragment thereof. There are two major advantages for the joining of the cFc with the ECD which are: (i) it extends the in vivo half-life of the fusion protein and (ii) it assists in the purification of the fusion proteins by affinity chromatography. Accordingly, the ECD of either IL-4Rα, IL-13Rα1, or IL-13Rα2, can be fused/joined with a canine IgG hinge region and a canine IgG (cFc). In certain alternatives the resulting fusion protein comprises in N-terminal to C-terminal order: the ECD of cIL-13Rα1, or cIL-13Rα2, or cIL-4Rα, a canine hinge region, and a cFc. WO 01/77332 discloses Fc fusion proteins containing IL-13Rα2 and canine IgG Fc sequences. However, these proteins contain an insertion of a non-self glycine residue (G) as a linker in between the ECD of IL-13Rα2 and the canine IgG Fc followed by 9 amino acid residues from the CHI domain of the canine IgG. Neither the glycyl linker nor the stretch of 9 amino acid residues from the CHI domain is present in the cFc fusion proteins of the present invention. The presence of the glycine residue followed by serine residue as in the Fc fusion proteins disclosed in WO 01/77332 creates an opportunity for enzymatic glycosylation of the fusion protein when it is expressed in cell culture systems and thereby could lead to the generation of variant molecules with some level of glycosylation on the serine residue. This would be undesirable from a manufacturability standpoint on an industrial scale. In addition, insertion of a glycine residue that is not part of the native canine IgG sequence creates the possibility of creating neoepitopes that could be recognized by the dog's immune system and stimulate the production of antibodies against the fusion proteins. Such antibodies could nullify the therapeutic utility of the fusion proteins.

Therefore, one advantage of particular cFc fusion proteins of the present invention is that they do not introduce non-self amino acid linkers and thereby, minimize the chance of leading to ADA in the in the treated animals. Additionally, the cFc fusion proteins of the present invention are maintained as non-contiguous molecules separating the cIL-4Rα Fc fusion protein from the canine IL-13Rα1 or canine IL-13Rα2 Fc fusion proteins. Although with some exceptions, 1 the absence of non-self amino acid linkers that connect fused domains are generally accepted to lead to low yields, low potency, and/or misfolding of the fused protein domains, surprisingly fusion proteins comprising the ECD of cIL-4 Rα, or cIL-13Rα1, or cIL-13Rα2, with a canine hinge region, and a cFc were successfully produced and purified even though they did not use non-self amino acid linkers. Accordingly, as shown below, these fusion proteins proved to be of high therapeutic value and have diminished ADA risk.

In direct contrast, in the studies described below, bispecific heterodimeric fusion proteins were found to lead to decreased expression levels, decreased stability, and decreased purity. In addition, as indicated above, they also may increase the potential of ADA formation in an animal subject. Moreover, it is not clear whether it will be necessary to use twice as much of the bispecific fusion protein to obtain the same therapeutic effect as that achieved from the combination of the two individual monospecific molecules (i.e., homodimers). Furthermore, the ability to control the efficacy/safety balance of the two individual functional components is lost, such as the ability to vary the dosage of one of the individual monospecific proteins, while keeping the dosage of the other constant.

In summary, it was found that the bispecific Fc fusion proteins had difficulties being expressed and being purified. More importantly, they were found to be less potent as an inhibitor of the cIL-4 and cIL-13 activity than the combination of two homodimers, particularly a cIL-4Rα-cFc homodimer together with a cIL-13Rα2-cFc homodimer (see the Examples below). Therefore, the present invention provides compositions comprising potent blockers of cIL-4 and cIL-13 activity i.e., the combination of homodimers of cIL-4Rα-cFc with cIL-13Rα2-cFc.

Moreover, in response to the need for better therapies for atopic dermatitis, the present invention also provides formulations and methodologies that can achieve the simultaneous modulation of the cIL-4/cIL-13, and cIL-31 signaling pathways involved in atopic dermatitis to produce a rapid onset of antipruritic action concomitant with a significant effect on the skin ¹ Fc fusion proteins comprising certain human proteins, e.g., human TNFR-Fc known as ENBREL® and human CTLA-4-Fc known as BELATACEPT®, do not include linkers. inflammation and an improvement in skin barrier function. These formulations combine the use of homodimers of cIL-4Rα-cFc fusion proteins and cIL-13Rα2-cFc fusion proteins, along with caninized rat antibodies that bind canine IL-31Rα.

Accordingly, the present invention provides compositions of homodimers of cFc fusion proteins that bind to either cIL-4 or cIL-13 and block the binding of these cytokines to their respective receptors. In addition, the present invention provides compositions that further comprise canine or caninized antibodies that bind cIL-31 or cIL-31R and block the binding of cIL-31 to the cIL-31 receptor. These compositions can be used to treat atopic dermatitis in canines.

### Abbreviations

Throughout the detailed description and examples of the invention the following abbreviations will be used:
ADCC Antibody-dependent cellular cytotoxicity
CDC Complement-dependent cyotoxicity
CDR Complementarity determining region in the immunoglobulin variable regions, defined using the Kabat numbering system cFc Canine fragment crystallizable region
CHO Chinese hamster ovary
EC50 concentration resulting in 50% efficacy or binding
ECD Extracellular domain
ELISA Enzyme-linked immunosorbant assay
FR Antibody framework region: the immunoglobulin variable regions excluding the CDR regions.
HRP Horseradish peroxidase
IC50 concentration resulting in 50% inhibition
IgG Immunoglobulin G
Kabat An immunoglobulin alignment and numbering system pioneered by Elvin A. Kabat [Sequences of Proteins of Immunological Interest, 5th Ed. Public Health Service, National Institutes of Health, Bethesda, Md. (1991)]
mAb Monoclonal antibody (also Mab or MAb)
PCR Polymerase chain reaction
PK Pharmacokinetics
V region The segment of IgG chains which is variable in sequence between different antibodies.
VH Immunoglobulin heavy chain variable region
VL Immunoglobulin light chain variable region
Vl Immunoglobulin lambda light chain variable region
Vk Immunoglobulin kappa light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment”, as it applies to an animal, e.g., a canine subject, cell, tissue, organ, or biological fluid, refers to contact of an exogenous pharmaceutical, therapeutic, diagnostic agent, or composition to the animal e.g., a canine subject, cell, tissue, organ, or biological fluid. Treatment of a cell encompasses contact of a reagent to the cell, as well as contact of a reagent to a fluid, where the fluid is in contact with the cell.

“Administration” and “treatment” also mean in vitro and ex vivo treatments, e.g., of a cell, by a reagent, diagnostic, binding compound, or by another cell. The term “subject” includes any organism, preferably an animal, more preferably a mammal (e.g., canine, feline, or human) and most preferably a canine.

“Treat” or “treating” means to administer a therapeutic agent, such as a composition comprising cFc fusion proteins of the present invention, internally or externally to e.g., a canine subject or patient having one or more symptoms, or being suspected of having a condition, for which the agent has therapeutic activity.

Typically, the therapeutic agent is administered in an amount effective to alleviate and/or ameliorate one or more disease/condition symptoms in the treated subject or population, whether by inducing the regression of or inhibiting the progression of such symptom(s) by any clinically measurable degree. The amount of a therapeutic agent that is effective to alleviate any particular disease/condition symptom (also referred to as the “therapeutically effective amount”) may vary according to factors such as the disease state, age, and weight of the patient (e.g., canine), and the ability of the pharmaceutical composition to elicit a desired response in the subject. Whether a disease/condition symptom has been alleviated or ameliorated can be assessed by any clinical measurement typically used by veterinarians or other skilled healthcare providers to assess the severity or progression status of that symptom. While an embodiment of the present invention (e.g., a treatment method or article of manufacture) may not be effective in alleviating the target disease/condition symptom(s) in every subject, it should alleviate the target disease/condition symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary (e.g., canine), or research subject, refers to therapeutic treatment, as well as research and diagnostic applications. “Treatment” as it applies to a human, veterinary (e.g., canine), or research subject, or cell, tissue, or organ, encompasses contact of the antibodies and/or fusion proteins of the present invention to e.g., a canine or other animal subject, a cell, tissue, physiological compartment, or physiological fluid.

As used herein, the term “feline” refers to any member of the Felidae family. Members of this family include wild, zoo, and domestic members, including domestic cats, pure-bred and/or mongrel companion cats, show cats, laboratory cats, cloned cats, and wild or feral cats.

As used herein, the term “canine” includes all domestic dogs, Canis lupus familiaris or Canis familiaris, unless otherwise indicated.

There are four known IgG heavy chain subtypes of canine IgG and two known light chain subtypes. The four IgG heavy chains are referred to as A, B, C, and D. These heavy chains represent four different subclasses of dog IgG, which are referred to as IgG-A (or IgGA), IgG-B (or IgGB), IgG-C (or IgGC) and IgG-D (or IgGD). Each heavy chain consists of one variable domain (VH) and three constant domains referred to as CH1, CH2, and CH3. The CHI domain is connected to the CH2 domain via an amino acid sequence referred to as the “hinge” or alternatively as the “hinge region”. The DNA and amino acid sequences of these four heavy chains IgGs were first identified by Tang et al. [Vet. Immunol. Immunopathol. 80:259-270 (2001)]. The amino acid and DNA sequences for these heavy chains IgGs are also available from the GenBank data bases. For example, the amino acid sequence of IgG-A heavy chain has accession number AAL35301.1, IgG-B has accession number AAL35302.1, IgG-C has accession number AAL35303.1, and IgG-D has accession number (AAL35304.1). Canine antibodies also contain two types of light chains, kappa and lambda. The DNA and amino acid sequence of these light chains can be obtained from GenBank Databases. For example, the kappa light chain amino acid sequence has accession number ABY 57289.1 and the lambda light chain has accession number ABY 55569.1.

The “fragment crystallizable region” abbreviated as “Fc region” or just “Fc” corresponds to the CH2-CH3 portion of an antibody that interacts with cell surface receptors called Fc receptors. As used herein a “canine fragment crystallizable region” is interchangeably abbreviated as “cFc region” or just “cFc” and corresponds to a canine fragment crystallizable region from a canine antibody. The canine fragment crystallizable region (cFc) of each of the four canine IgGs were first described by Tang et al. [Vet. Immunol. Immunopathol. 80:259-270 (2001); see also, Bergeron et al., Vet. Immunol. Immunopathol. 157:31-41 (2014)].

As used herein, the “extracellular domain” or “ECD” of a transmembrane interleukin, such as canine Interleukin-4 receptor alpha, canine Interleukin-13 receptor alpha 1, or canine Interleukin-13 receptor alpha 2, refers to the portion of the Interleukin protein that naturally projects into the environment surrounding the cell. The ECD does not include the transmembrane portion of the interleukin. The ECD of canine Interleukin-4 receptor alpha binds to canine IL-4. The ECD of canine Interleukin-13 receptor alpha 1 and canine Interleukin-13 receptor alpha 2 both bind to IL-13.

As used herein, an “artificial protein” and an “artificial protein molecule” are used interchangeably and denote a protein (or multimer of proteins, such as dimers, heterodimers, tetramers, and heterotetramers, etc.) that does not naturally exist in nature, such as a man-made fusion protein.

As used herein a “fusion protein” is an artificial protein that comprises amino acid sequences from two or more different proteins which are joined together by peptide bonds.

As used herein a “cFc fusion protein” is an artificial protein that joins the cFc of an IgG antibody, which can include a hinge region, e.g., the IgGB hinge region-CH2-CH3, with another biologically active protein domain to generate a molecule with unique structure and therapeutic utility. For example, a canine IL-13Rα2-cFc fusion protein comprises the extracellular domain (ECD) of canine IL-13Rα2 linked to the N-terminus of a canine IgG Fc (cFc). The ECD of the IL-13Rα2 may be linked to the N-terminus of the cFc by a canine hinge region. The cFc fusion proteins of the present invention, although exemplified by the use of the IgGB hinge region and the IgGB cFc, are in no way so limited, but rather they include the corresponding fusion proteins with the cFcs of IgGA, IgGC, and IgGD and optionally the hinge regions of IgGA, IgGC, and IgGD. Accordingly, the canine Fc fusion protein cIL-4Rα-cIgGB-Fc is one species of the cIL-4Rα-cFc genus, which also includes cIL-4Rα-cIgGA-Fc, cIL-4Rα-cIgGC-Fc, and cIL-4Rα-cIgGD-Fc.

A particular component of a cFc fusion protein of the present invention (e.g., a canine ECD, a canine hinge region, or a cFc) that “comprises an amino acid sequence that is identical to amino acid sequence of a protein naturally found in canines” consists of an amino acid sequence that is identical to the corresponding amino acid sequence of a region of a corresponding protein found in canines, including naturally occurring variants thereof. For example, when the component of the cFc fusion protein is the cFc itself, and the cFc “comprises an amino acid sequence that is identical to amino acid sequence of a protein naturally found in canines”, the amino acid sequence of the cFc region of the cFc fusion protein is identical to that of a naturally occurring canine cFc region of a canine antibody, or variant thereof.

As used herein, a cFc fusion protein that is “composed solely of amino acid sequences that are identical to amino acid sequences of proteins naturally found in canines” solely consists of components of that cFc fusion protein that consist of amino acid sequences that are individually identical to the amino acid sequences of the corresponding region of proteins found in canines, including naturally occurring variants thereof. For example, when the cFc fusion protein is a cIL-13Rα2-cFc fusion protein that consists of three components: an ECD of a cIL-13Rα2 linked to the N-terminus of a cFc by a canine hinge region, and is “composed solely of amino acid sequences that are identical to amino acid sequences of proteins naturally found in canines” the individual amino acid sequences of all three components of the cIL-13Rα2-cFc fusion protein: (i) the amino acid sequence of the ECD of the cIL-13Rα2, (ii) the amino acid sequence of the cFc, and (iii) the amino acid sequence of the canine hinge region, are individually identical to the amino acid sequence of the corresponding region of proteins naturally found in canines, including naturally occurring variants thereof.

As used herein the term “sole linker” of a cFc fusion protein of the present invention indicates that the linker is the only linker in that cFc fusion protein. For example, if that canine hinge region is the only linker comprised by a cFc fusion protein comprising an ECD of the cIL-13Rα2 linked to the N-terminus of the cFc by a canine hinge region, then that canine hinge region is a sole linker.

As used herein a “canine Interleukin-13 receptor alpha 1-canine fragment crystallizable region fusion protein”, “canine Interleukin-13 receptor alpha 1-cFc fusion protein”, “canine IL-13Rα1-cFc fusion protein”, or “cIL-13Rα1-cFc fusion protein” are all used interchangeably and comprise the extracellular domain (ECD) of cIL-13Rα1 [or fragment of the ECD that binds canine Interleukin-13 (cIL-13)] connected to a canine IgG Fc (cFc) via a peptide linkage. In particular embodiments, a cIL-13Rα1-cFc fusion protein further comprises a canine hinge region that links the ECD of the cIL-13Rα1 (or fragment of the ECD that binds cIL-13) to the cFc. The cIL-13Rα1-cFc fusion protein can be generated from a chemically synthesized nucleic acid encoding the cIL-13Rα1 ECD (or fragment of the ECD that binds cIL-13) with the cFc (either with or without the linking hinge region) through genetic engineering.

As used herein a “canine Interleukin-13 receptor alpha 2-canine fragment crystallizable region fusion protein”, “canine Interleukin-13 receptor alpha 2-cFc fusion protein”, “canine IL-13Rα2-cFc fusion protein” or “cIL-13Rα2-cFc fusion protein” are all used interchangeably and comprise the extracellular domain (ECD) of cIL-13Rα2 [or fragment of the ECD that binds canine Interleukin-13 (cIL-13)] connected to a canine IgG Fc (cFc) via a peptide linkage. In particular embodiments, a cIL-13Rα2-cFc fusion protein further comprises a canine hinge region that links the ECD of the cIL-13Rα2 (or fragment of the ECD that binds cIL-13) to the cFc. The cIL-13Rα2-cFc fusion protein can be generated from a chemically synthesized nucleic acid encoding the cIL-13Rα2 ECD (or fragment of the ECD that binds cIL-13) with the cFc (either with or without the linking hinge region) through genetic engineering.

As used herein a “canine Interleukin-4 receptor alpha-canine fragment crystallizable region fusion protein”, “canine Interleukin-4 receptor alpha-cFc fusion protein”, “canine IL-4Rα-cFc fusion protein” or “cIL-4Rα-cFc fusion protein” are all used interchangeably and comprise the extracellular domain (ECD) of cIL-4Rα [or fragment of the ECD that binds canine Interleukin-4 (cIL-4)] connected to a canine IgG Fc (cFc) via a peptide linkage. In particular embodiments, a cIL-4Rα-cFc fusion protein further comprises a canine hinge region that links the ECD of the cIL-4Rα (or fragment of the ECD that binds cIL-4) to the cFc. The cIL-4Rα-cFc fusion protein can be generated from a chemically synthesized nucleic acid encoding the cIL-4Rα ECD (or fragment of the ECD that binds cIL-4) with the cFc (either with or without the linking hinge region) through genetic engineering.

As used herein a cIL-4Rα-cFc fusion protein comprising a “fragment of an ECD of cIL-4Rα that binds cIL-4” (or interchangeably, a “fragment thereof” of an ECD of the cIL-4Rα that binds cIL-4), has a binding affinity for cIL-4 that is at most a factor of 100 less than the binding affinity of the corresponding cIL-4Rα-cFc fusion protein comprising the full length ECD, i.e., the dissociation constant is at most a factor of 102 higher (e.g., 10⁻⁷M as compared to 10⁻⁹M). In certain embodiments, a cIL-4Rα-cFc fusion protein comprising a fragment of an ECD of cIL-4Rα that binds cIL-4 has a binding affinity for cIL-4 that is at most a factor of 10 less than the binding affinity of the corresponding cIL-4Rα-cFc fusion protein comprising the full length ECD, i.e., the dissociation constant is at most a factor of 10 higher. In still other embodiments, a cIL-4Rα-cFc fusion protein comprising a fragment of an ECD of cIL-4Rα that binds cIL-4 has a binding affinity for cIL-4 that is at most a factor of 5 less than that of the binding affinity of the corresponding cIL-4Rα-cFc fusion protein comprising the full length ECD, i.e., the dissociation constant is at most a factor of 5 higher.

As used herein a cIL-13Rα2-cFc fusion protein comprising a “fragment of an ECD of cIL-13Rα2 that binds cIL-13” (or interchangeably, “a fragment thereof” of an ECD of the cIL-13Rα2 that binds cIL-13), has a binding affinity for cIL-13 that is at most a factor of 100 less than the binding affinity of the corresponding cIL-13Rα2-cFc fusion protein comprising the full length ECD, i.e., the dissociation constant is at most a factor of 102 higher. In certain embodiments, a cIL-13Rα2-cFc fusion protein comprising a fragment of an ECD of cIL-13Rα2 that binds cIL-13 has a binding affinity for cIL-13 that is at most a factor of 10 less than the binding affinity of the corresponding cIL-13Rα2-cFc fusion protein comprising the full length ECD, i.e., the dissociation constant is at most a factor of 10 higher. In still other embodiments, a cIL-13Rα2-cFc fusion protein comprising a fragment of an ECD of cIL-13Rα2 that binds cIL-13 has a binding affinity for cIL-13 that is at most a factor of 5 less than that of the binding affinity of the corresponding cIL-13Rα2-cFc fusion protein comprising the full length ECD, i.e., the dissociation constant is at most a factor of 5 higher.

As used herein a cIL-13Rα1-cFc fusion protein comprising a “fragment of an ECD of cIL-13Rα1 that binds cIL-13” (or interchangeably, “a fragment thereof” of the ECD of cIL-13Rα1 that binds cIL-13), has a binding affinity for cIL-13 that is at most a factor of 100 less than the binding affinity of the corresponding cIL-13Rα1-cFc fusion protein comprising the full length ECD, i.e., the dissociation constant is at most a factor of 102 higher. In certain embodiments, a cIL-13Rα1-cFc fusion protein comprising a fragment of an ECD of cIL-13Rα1 that binds cIL-13 has a binding affinity for cIL-13 that is at most a factor of 10 less than the binding affinity of the corresponding cIL-13Rα1-cFc fusion protein comprising the full length ECD, i.e., the dissociation constant is at most a factor of 10 higher. In still other embodiments, a cIL-13Rα1-cFc fusion protein comprising a fragment of an ECD of cIL-13Rα1 that binds cIL-13 has a binding affinity for cIL-13 that is at most a factor of 5 less than that of the binding affinity of the corresponding cIL-13Rα1-cFc fusion protein comprising the full length ECD, i.e., the dissociation constant is at most a factor of 5 higher.

As used herein a “homodimer” of a canine Interleukin receptor-cFc fusion protein of the present invention is a dimer of two monomeric fusion proteins that minimally have the same ECD (or a fragment of that ECD that binds the corresponding ligand). The two monomeric fusion proteins generally also have the same cFc and the same hinge region. For example, when the canine Interleukin receptor-cFc fusion protein is a cIL-4Rα-cFc fusion protein, the ECD is an IL-4Rα ECD and the ligand is cIL-4. The two monomers of the homodimers are held together by disulfide bonds formed by the cysteine residues in the hinge region of each monomer. For example, a homodimer of a cIL-4Rα-cFc fusion protein comprises two cIL-4Rα-cFc fusion protein monomers and a homodimer of a cIL-13Rα2-cFc fusion protein comprises two cIL-13Rα2-cFc fusion protein monomers.

As used herein a “heterodimer” of canine Interleukin receptor-cFc fusion proteins of the present invention is a dimer of two monomeric fusion proteins that have different ECDs (or fragments of the respective ECDs that bind the corresponding ligand of the respective ECD). The two monomeric fusion proteins generally have the same cFc, although in certain instances they can be slightly different due to modifications to keep the two monomers together. For example, a heterodimer of a cIL-4Rα-cFc fusion protein and a cIL-13Rα2-cFc fusion protein comprises one cIL-4Rα-cFc fusion protein monomer and one cIL-13Rα2-cFc fusion protein monomer, whereas a heterodimer of a cIL-4Rα-cFc fusion protein and a cIL-13Rα1-cFc fusion protein comprises one cIL-4Rα-cFc fusion protein monomer and one cIL-13Rα1-cFc fusion protein monomer. One such embodiment is cIL-4Rα-13Rα1 ZW1-cFc, which is a heterodimer of cIL-4Rα-cFc-ZW-A and cIL-13Rα1-cFc-ZW-B. Another such embodiment is cIL-4Rα-13Rα2 ZW1-cFc, which is a heterodimer of cIL-4Rα-cFc-ZW-A and cIL-13Rα2-cFc-ZW-B.

As used herein, the term “antibody” refers to any form of antibody that exhibits the desired biological activity. An antibody can be a monomer, dimer, or larger multimer. Thus, it is used in the broadest sense and specifically covers, but is not limited to, monoclonal antibodies (including full length monoclonal antibodies), polyclonal antibodies, multi-specific antibodies (e.g., bispecific antibodies), caninized antibodies, fully canine antibodies, chimeric antibodies and camelized single domain antibodies. “Parental antibodies” are antibodies obtained by exposure of an immune system to an antigen prior to modification of the antibodies for an intended use, such as caninization of an antibody for use as a canine therapeutic antibody.

As used herein, cFc fusion proteins of the present invention or antibodies used in the present invention that “block” or is “blocking” or is “blocking the binding” of, e.g., a canine receptor to its binding partner (ligand), is an antibody and/or fusion protein that blocks (partially or fully) the binding of the canine receptor to its canine ligand and vice versa, as determined in standard binding assays (e.g., BIACore®, ELISA, or flow cytometry).

Typically, an antibody or antigen binding fragment of the invention retains at least 10% of its canine antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen binding affinity as the parental antibody. It is also intended that an antibody or antigen binding fragment of the invention can include conservative or non-conservative amino acid substitutions (referred to as “conservative variants” or “function conserved variants” of the antibody) that do not substantially alter its biologic activity.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81:6851-6855 (1984)]. Typically the variable domains are obtained from an antibody from an experimental animal (the “parental antibody”), such as a rodent, and the constant domain sequences are obtained from the animal subject antibodies, e.g., human or canine so that the resulting chimeric antibody will be less likely to elicit an adverse immune response in a human or canine subject respectively, than the parental (e.g., rodent) antibody.

As used herein, the term “caninized antibody” refers to forms of antibodies that contain sequences from both canine and non-canine (e.g., rat) antibodies. In general, the caninized antibody will comprise substantially all of at least one or more typically, two variable domains in which all or substantially all of the hypervariable loops correspond to those of a non-canine immunoglobulin (e.g., comprising 6 CDRs as exemplified below), and all or substantially all of the framework (FR) regions (and typically all or substantially all of the remaining frame) are those of a canine immunoglobulin sequence. As exemplified herein, a caninized antibody comprises both the three heavy chain CDRs and the three light chain CDRS from a rat anti-canine antigen antibody together with a canine frame or a modified canine frame. A modified canine frame comprises one or more amino acids changes as exemplified herein that further optimize the effectiveness of the caninized antibody, e.g., to increase its binding to its canine antigen and/or its ability to block the binding of that canine antigen to the canine antigen's natural binding partner. Caninized murine or rat anti-canine antibodies that bind canine IL-31 and IL-31R alpha include but are not limited to antibodies for use in the present invention that comprise canine IgGA, IgGB, IgGC, or IgGD heavy chains.

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” [i.e. CDRL1 (or LCDR1), CDRL2 (or LCDR2), and CDRL3 (or LCDR3) in the light chain variable domain and CDRHI (or HCDR1), CDRH2 (or HCDR2), and CDRH3 (or HCDR3) in the heavy chain variable domain]. [See Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196:901-917 (1987) defining the CDR regions of an antibody by structure].

As used herein, the term “framework” or “FR” residues refers to those variable domain residues other than the hypervariable region residues defined herein as CDR residues.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse or rat antibody) in both chains. Optionally the heavy and/or light chains of the canine antibody may contain some foreign non-CDR residues, e.g., so as to preserve the conformation of the foreign CDRs within the canine antibody, and/or to modify the Fc function, as exemplified below and/or disclosed in U.S. Pat. No. 10,106,607 B2.

As used herein an “antipruritic agent” is a compound, macromolecule, and/or formulation that tends to inhibit, relieve, and/or prevent itching. Antipruritic agents are colloquially referred to as anti-itch drugs.

As used herein an “antipruritic antibody” is an antibody that can act as an antipruritic agent in an animal, including a mammal such as a human, a canine, and/or a feline, particularly with respect to atopic dermatitis. In particular embodiments, the antipruritic antibody binds to specific proteins in the IL-31 signaling pathway, such as IL-31 or its receptor IL-31Rα. The binding of the antipruritic antibody to its corresponding antigen (e.g., IL-31 or IL-31Rα) inhibits the binding of e.g., IL-31 with IL-31Rα, and interferes with and/or prevents the successful signaling of this pathway, and thereby inhibits, relieves, and/or prevents the itching that is otherwise caused by the IL-31 signaling pathway.

As used herein an “anti-inflammatory agent” is a compound, macromolecule, and/or formulation that that reduces inflammation by blocking the interaction of certain substances in the body that cause inflammation. The anti-inflammatory agent can be a cFc fusion protein that can act as an anti-inflammatory agent in an animal, including a mammal such as a human, a canine, and/or a feline, particularly with respect to atopic dermatitis. In particular embodiments, the anti-inflammatory cFc fusion protein binds to specific proteins in the IL-4/IL-13 signaling pathway, such as IL-4 or IL-13. The binding of the anti-inflammatory cFc fusion protein to its corresponding antigen (e.g., IL-4) inhibits the binding of e.g., IL-4 with IL-4Rα, and interferes with and/or prevents the signaling of this pathway, thereby interfering with or preventing the chronic inflammation associated with atopic dermatitis. The combination of homodimers of the cIL-4Rα-cFc fusion protein with homodimers of the cIL-13Rα2-cFc fusion protein acts as an anti-inflammatory agent in the treatment of atopic dermatitis.

As used herein a “bispecific fusion protein” is an artificial protein that either can be a contiguous protein, e.g., two different biologically active protein domains joined together via peptide bonds, e.g., the ECD of cIL-4Rα, the ECD of cIL-13Rα1, together with a cFc and optional linkers. Alternatively, bispecific fusion protein can be a heterodimer fusion protein in which the two different biologically active protein domains are individually joined together with a fusion partner via peptide bonds, but joined together in the heterodimer fusion protein by non-peptide bonds, which can be either covalent or noncovalent bonds. For example, a heterodimer formed by combining two monomeric fusion proteins that have different ECDs such as a heterodimer of a cIL-4Rα-cFc fusion protein monomer and a cIL-13Rα2-cFc fusion protein monomer.

Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared ×100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In particular embodiments, selected deletions or insertions that could otherwise alter the correspondence between the two amino acid sequences are taken into account.

Sequence similarity includes identical residues and nonidentical, biochemically related amino acids. Biochemically related amino acids that share similar properties and may be interchangeable.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes frequently can be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table A directly below.

TABLE A
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the cFc fusion proteins of the invention are also contemplated by the present invention. “Function-conservative variants,” as used herein, refers to the cFc fusion proteins in which one or more amino acid residues have been changed without altering a desired property, such an antigen affinity and/or specificity. Such variants include, but are not limited to, replacement of an amino acid with one having similar properties, such as the conservative amino acid substitutions of Table A above.

### Nucleic Acids

The present invention comprises the cFc fusion proteins of the present invention and compositions that comprise the cFc fusion proteins of the present invention along with the antibodies used in the present invention (see e.g., Examples below).

Also included in the present invention are the nucleic acids that encode the cFc fusion proteins provided and the immunoglobulin polypeptides used in the present invention, comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the caninized antibodies provided herein when the comparison is performed by a BLAST algorithm wherein the parameters of the algorithm are selected to give the largest match between the respective sequences over the entire length of the respective reference sequences. The present invention further provides nucleic acids that encode the fusion proteins and/or the immunoglobulin polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm, wherein the parameters of the algorithm are selected to give the largest match between the respective sequences over the entire length of the respective reference sequences, are also included in the present invention.

As used herein, nucleotide and amino acid sequence percent identity can be determined using C, Mac Vector (Mac Vector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141 (1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70 (1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

The cFc fusion proteins of the present invention (and antibodies used in the present invention) can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application Ser. No. 89/303,964.4

### Pharmaceutical Compositions and Administration

To prepare pharmaceutical or sterile compositions comprising the cFc fusion proteins of the present invention, either alone or with the antibodies used in the present invention, can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, pharmaceutical compositions comprising the cFc fusion proteins of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED50 with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, pharmaceutical compositions comprising the cFc fusion proteins of the present invention can be administered by an invasive route such as by injection. In further embodiments of the invention, pharmaceutical compositions comprising the cFc fusion proteins of the present invention are administered intravenously, subcutaneously, intramuscularly, intraarterially, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399, 163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer compositions comprising the cFc fusion proteins of the present invention (and optionally the antibodies used in the present invention) in a local rather than systemic manner, often in a depot or sustained release formulation.

The administration regimen depends on several factors, including the serum or tissue turnover rate of the therapeutic the antibodies, and/or cFc fusion proteins, the level of symptoms, the immunogenicity of the therapeutic antibodies and/or cFc fusion proteins and the accessibility of the target cells in the biological matrix. Preferably, the administration regimen delivers sufficient therapeutic antibodies and/or cFc fusion proteins to effect improvement in the target disease/condition state, while simultaneously minimizing undesired side effects. Accordingly, the amount of biologic delivered depends in part on the particular therapeutic antibodies, and/or fusion proteins and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of the symptoms.

The compositions comprising the cFc fusion proteins of the present invention, either alone or with the antibodies used in the present invention may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2μg/kg, 0.5μg/kg, 1μg/kg, 10μg/kg, 100μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of cFc fusion proteins of the present invention in the canine's serum, such as 0.1, 0.3, 1, 3, 10, 30, 100, 300 μg/ml or more. In other embodiments, the cFc fusion proteins of the present invention are administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder or condition and/or a reduction in the severity of the symptoms of such disorder or condition. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject (e.g., a canine) with a disorder, condition and/or symptom, or with the potential to develop such a disorder, disease or symptom.

As used herein, the terms “therapeutically effective amount”, “therapeutically effective dose” and “effective amount” refer to an amount of the cFc fusion proteins of the present invention that, when administered alone or in combination with an additional therapeutic agent to a cell, tissue, or subject, e.g., canine, is effective to cause a measurable improvement in one or more symptoms of a disease or condition or the progression of such disease or condition. A therapeutically effective dose further refers to that amount of the antibodies and/or fusion proteins sufficient to result in at least partial amelioration of symptoms, e.g., treatment, healing, prevention or amelioration of the relevant medical condition, or an increase in rate of treatment, healing, prevention or amelioration of such conditions. When applied to a combination, a therapeutically effective dose refers to combined amounts of the active ingredients that result in the therapeutic effect, whether administered in combination, serially, or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess severity of the condition.

### Other Combination Therapies

The compositions comprising cFc fusion proteins of the present invention (with or without an antibody used in the present invention), can comprise one or more additional therapeutic component. One such family of therapeutic components are Janus kinase (JAK) inhibitors. In a particular embodiment of this type the JAK inhibitor comprises the chemical formula of:

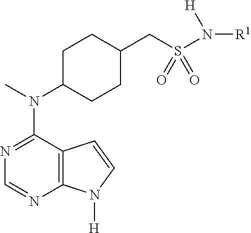

where R¹ is C₁₋₄ alkyl optionally substituted with hydroxy, and pharmaceutically acceptable salts thereof [U.S. Pat. Nos. 8,133,899; 8,987,283]. More particularly the JAK inhibitor is oclacitinib and even more particularly, oclacitinib maleate.

An alternative JAK inhibitor, which preferentially inhibits JAK1 relative to JAK3 is: 1-[(3R,4S)-4-cyanotetrahydropyran-3-yl]-3-[(2-fluoro-6-methoxy-4-pyridyl)amino]pyrazole-4-carboxamide, which comprises the chemical formula of:

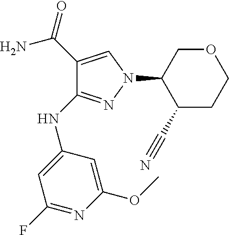 [SMILES: COc1cc(Nc2nn([C@H]3COCC[C@@H]3C#N)cc2C(N)=O)cc(F)n1]

and pharmaceutically acceptable salts thereof [see, WO 2018/108969].

Yet another alternative JAK inhibitor, is 3-Azetidineacetonitrile, 1-(cyclopropylsulfonyl)-3-[4-(7H-pyrrolo[2,3-d]pyrimidin-4-yl)-1H-pyrazol-1-yl]—(Source: CAS); also referred to as {1-(cyclopropanesulfonyl)-3-[4-(7H-pyrrolo[2,3-d]pyrimidin-4-yl)-1H-pyrazol-1-yl]azetidin-3-yl}acetonitrile (Source: USAN Program chemical consultant), which comprises the chemical formula of:

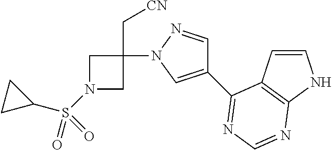 [SMILES: N#CCC1(n2cc(-c3ncnc4[nH]ccc34)cn2)CN(S(=O)(=O)C2CC2)C1]

and pharmaceutically acceptable salts thereof [see, US 2020/0339585].

Another therapeutic component that can be added to a composition of the present invention can be a spleen tyrosine kinase (SYK) inhibitor. One such SYK inhibitor is (1S,4R)-4-hydroxy-2,2-dimethyl-4-{5-[3-methyl-5-(4-methyl-pyrimidin-2-ylamino)-phenyl]-1,3-thiazol-2-yl}-cyclohexanecarboxylic acid or pharmaceutically acceptable salts thereof [see e.g., U.S. Pat. No. 8,759,366].

In addition, yet another therapeutic component that can be added to a composition of the present invention can an antagonist to a chemoattractant receptor-homologous molecule expressed on TH2 cells comprising the chemical formula of:

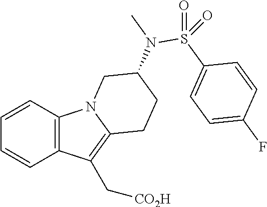 [SMILES: CN([C@@H]1CCc2c(CC(=O)O)c3ccccc3n2C1)S(=O)(=O)c1ccc(F)cc1]

and pharmaceutically acceptable salts thereof [see also, U.S. Pat. Nos. 7,696,222, 8,546,422, 8,637,541, WO 2010/099039; WO 2010/031183; and U.S. Pat. No. 8,546,422].

These additional therapeutic components can be administered to the canine subject prior to, in conjunction with, or following the administration of the composition comprising the antibodies, and/or fusion proteins of the present invention.

The magnitude of prophylactic or therapeutic dose of the JAK inhibitors, SYK inhibitors, or chemoattractant receptor-homologous molecules listed above will, of course, vary with the nature and the severity of the condition to be treated and with the particular inhibitor and its route of administration. It will also vary according to a variety of factors including the age, weight, general health, sex, diet, time of administration, rate of excretion, drug combination and response of the individual canine. In general, the daily dose from about 0.001 mg to about 100 mg per kg body weight of the dog, preferably 0.01 mg to about 10 mg per kg. In another embodiment, the daily dose is from about 0.2 mg per kg to about 1.0 mg/kg of body weight of the dog. In another embodiment, the daily dose is from about 0.1 mg per kg to about 3.0 mg/kg of body weight of the dog. On the other hand, it may be necessary to use dosages outside these limits in some cases. The amount of active ingredient that may be combined with the carrier materials to produce a single dosage form will vary depending upon the host treated and the particular mode of administration. For example, a formulation intended for the oral administration may contain from 0.05 mg to 5 g of active agent compounded with an appropriate and convenient amount of carrier material which may vary from about 5 to about 99.95 percent of the total composition. Dosage unit forms will generally contain between from about 0.1 mg to about 0.4 g of an active ingredient, typically 0.5 mg, 1 mg, 2 mg, 5 mg, 10 mg, 25 mg, 50 mg, 100 mg, 200 mg, or 400 mg.

### EXAMPLES

### Example 1

### Homodimeric Fc Fusion Proteins

### Generating the Recombinant Fusion Proteins:

The recombinant fusion proteins listed in Tables 2B and 2C below, were obtained from a commercial manufacturer after providing the precise amino acid sequences for the selected fusion proteins to them. The amino acid sequences can be obtained from publicly available protein databases, such as GenBank, e.g., the accession numbers for the full length amino acid sequences include accession #XP_022275636.1 for Canis lupus familiaris interleukin-4 receptor subunit alpha isoform X1, accession #XP_038306633.1 for Canis lupus familiaris interleukin-13 receptor subunit alpha-1 isoform X2, and accession #NP_001003075.1 Canis lupus familiaris for interleukin-13 receptor subunit alpha-2 precursor. Typically, in order to produce these fusion proteins recombinantly, the DNA encoding the canine fusion proteins is chemically synthesized and then cloned into suitable expression vectors (e.g., the pcDNA3.4 expression vector) to produce the proteins in cells such as CHO or HEK-293 cells. Accordingly, the commercial manufacturer elects an optimal nucleotide sequence that encodes the amino acid sequence of the fusion protein, chemically synthesizes the nucleic acid, inserts the nucleic acid into an expression vector that produces the corresponding recombinant fusion protein, and then purifies the expressed fusion proteins. The nucleic acid sequence is typically produced at the commercial supplier in a process that entails the following steps:
1. Designing and synthesizing a number of oligonucleotides with a length of about 100 nucleotides, based on the target gene sequence (the synthesized overlapping oligonucleotides cover the ECD, the hinge region, and the cFc);
2. Assembling the oligonucleotides together to get a full-length gene sequence through the use of Polymerase Chain Reaction (PCR); and
3. Purifying the PCR product using a DNA gel extraction kit and used as an insert in the subsequent cloning step. In the present case, the recombinant fusion proteins were produced in CHO cells and purified using protein A column chromatography.

The nucleic acids encoding the cFc fusion proteins of the present invention comprise a coding sequence for the extracellular domain (ECD) or fragment thereof of a selected canine interleukin receptor, i.e., cIL-4Rα, cIL-13Rα1, or cIL-13Rα2, and a coding sequence for a canine IgG hinge region along with a canine IgG (cFc). The resulting fusion protein comprises in N-terminal to C-terminal order: the ECD, the hinge region (in bold), and the cFc. The cFc and hinge region can be derived from canine IgGA, IgGB, IgGC, or IgGD. The cFc fusion protein may optionally have amino acid replacements to allow for extended half-life in vivo or to eliminate some effector functions such as antibody-dependent cellular cytotoxicity (ADCC) or complement-mediated cytotoxicity (CDC) [see e.g., U.S. Pat. No. 10,106,607 B2].

The two monomers of the homodimers are held together by disulfide bonds formed by the cysteine residues in the hinge region of each monomer. Homodimeric proteins are made in separate host cells (such as CHO cells) and then may be combined after purification from their respective production cells. The homodimeric proteins can be administered to dogs via a variety of routes such as IV, SC, IP, or IM. Homodimeric proteins may be administered at doses ranging from 0.1 ug/kg to 20 mg/kg or more. Typically, homodimeric proteins may be administered at doses ranging from 0.1 mg/kg to 10 mg/kg.

Examples of the homodimeric Fc fusion proteins of the present invention are:

cIL-4Rα-cIgGB-Fc
[SEQ ID NO: 5]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLPPKREN
GRVPRPPDCPKCPAPEMLGGPSVFIFPPKPKDTLLIARTPEVTCVVVDLDPEDPEVQISWFVDGKQMQTAK
TQPREEQFNGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWORGDTFICAVM
HEALHNHYTQESLSHSPGK
cIL-13Rα1-cIgGB-Fc
[SEQ ID NO: 6]
GGVAAPTETQPPVTNLSVSVENLCTVIWTWDPPEGASPNCTLRYFSHEDNKQDKKIAPETHRSKEVPLNER
ICLQVGSQCSTNESDNPSILVEKCTPPPEGDPESAVTELQCVWHNLSYMKCTWLPGRNTSPDTNYTLYYWH
SSLGKILQCEDIYREGQHIGCSFALTNLKDSSFEQHSVQIVVKDNAGKIRPSFNIVPLTSHVKPDPPHIKR
LFFQNGNLYVQWKNPQNFYSRCLSYQVEVNNSQTETNDIFYVEEAKCONSEFEGNLEGTICFMVPGVLPDT
LNTVRIRVRINKLCYEDDKLWSNWSQAMSIGENTDPTPKRENGRVPRPPDCPKCPAPEMLGGPSVFIFPPK
PKDTLLIARTPEVTCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDVEWQSNGQQE
PESKYRTTPPQLDEDGSYFLYSKLSVDKSRWQRGDTFICAVMHEALHNHYTQESLSHSPGK
cIL-13Rα2-cIgGB-Fc
[SEQ ID NO: 7]
SMLSNAEIKVNPPQDFEIVDPGYLGYLSLQWQPPLEPDNEKECTIEYELKYRNIDSENWKTIITKNIHYKD
GFDLNKGIEAKINTLLPAQCTNGSEVRSSWAETTYWTSPQGNRETKIQDMDCVYYNWQYLVCSWKPGMGVH
FDTNYQLFYWYEGLDHSAECTDYIKVNGKNMGCREPYLESSDYKDFYICVNGSSESQPIRPSYFIFQLQNI
VKPMPPDYLSLTVKNSEEINLKWNMPKGPIPAKCFIYEIEFTEDGTTWVTTTVENEIQITRTSNESQKLCF
LVRSKVNIYCSDDGIWSEWSDEQCWKGDIWKETPKRENGRVPRPPDCPKCPAPEMLGGPSVFIFPPKPKDT
LLIARTPEVTCVVVDLDPEDPEVQISWFVDGKQMQTAKTQPREEQFNGTYRVVSVLPIGHQDWLKGKQFTC
KVNNKALPSPIERTISKARGQAHQPSVYVLPPSREELSKNTVSLTCLIKDFFPPDIDVEWQSNGQQEPESK
YRTTPPQLDEDGSYFLYSKLSVDKSRWQRGDTFICAVMHEALHNHYTQESLSHSPGK

### Example 2

Homodimeric Cfc Fusion Proteins with Extended Half-Life

Understanding the structure and characteristics of the interaction between the so-called neonatal Fc receptor (FcRn) and IgG antibodies has provided a basis for antibody or Fc engineering work that improves the serum half-lives of IgG antibodies and the Fc fusion proteins. Serum half-life extension of proteins and the mechanism behind approaches to prolong serum half-life of such proteins were described by several investigators [for example, see Ko et al., BioDrugs 35:147-157 (2021)]. Homodomeric proteins with extended half-life are synthesized and produced recombinantly from nucleotide sequences encoding the desired amino acid sequences as described in Example 1 above.

Examples of recombinant cFc fusion proteins with extended in vivo half-life are provided below. In these examples the canine cFc is IgGB, however the use of alternative cFc's, i.e., IgGA, IgGC, and IgGD in the cFc fusion proteins of the present invention also are part of the present invention.

Canine IgG-B Fc was first defined by Tang et al. [Vet Immunology & Immunopathology, 80:259-270 (2001)], as comprising the amino acid sequence of SEQ ID NO: 51, provided below.

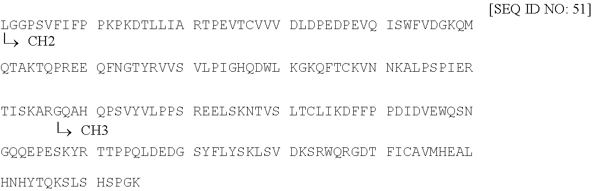

The amino acid sequence of the cFc portion of the recombinant fusion proteins include amino acid replacements (bold and underlined) that result in higher affinity binding to FcRn at mildly acidic pH (e.g., pH 6.0) than wild type cFc, while at the same time having similar binding affinity to FcRn at neutral pH (e.g., pH 7.0-7.2) as that exhibited by wild type cFc. The hinge region of each of the sequences is in bold, but not underlined.

cIL-4Rα-cIgGB-Fc-YTE
CH2 Numbering (L18Y/A20T/T22E)
[SEQ ID NO: 8]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLPPKREN
GRVPRPPDCPKCPAPEMLGGPSVFIFPPKPKDTLYITREPEVTCVVVDLDPEDPEVQISWFVDGKQMQTAK
TQPREEQFNGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWORGDTFICAVM
HEALHNHYTQESLSHSPGK
cIL-13Rαl-cIgGB-Fc-YTE
CH2 Numbering (L18Y/A20T/T22E)
[SEQ ID NO: 9]
GGVAAPTETQPPVTNLSVSVENLCTVIWTWDPPEGASPNCTLRYFSHFDNKQDKKIAPETHRSKEVPLNER
ICLQVGSQCSTNESDNPSILVEKCTPPPEGDPESAVTELQCVWHNLSYMKCTWLPGRNTSPDTNYTLYYWH
SSLGKILQCEDIYREGQHIGCSFALTNLKDSSFEQHSVQIVVKDNAGKIRPSFNIVPLTSHVKPDPPHIKR
LFFQNGNLYVQWKNPQNFYSRCLSYQVEVNNSQTETNDIFYVEEAKCONSEFEGNLEGTICFMVPGVLPDT
LNTVRIRVRTNKLCYEDDKLWSNWSQAMSIGENTDPTPKRENGRVPRPPDCPKCPAPEMLGGPSVFIFPPK
PKDTLYITREPEVTCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDVEWQSNGQQE
PESKYRTTPPQLDEDGSYFLYSKLSVDKSRWQRGDTFICAVMHEALHNHYTQESLSHSPGK
CIL-13Rα2-cIgGB-Fc-YTE
CH2 Numbering (L18Y/A20T/T22E)
[SEQ ID NO: 10]
SMLSNAEIKVNPPQDFEIVDPGYLGYLSLOWQPPLFPDNFKECTIEYELKYRNIDSENWKTIITKNIHYKD
GFDLNKGIEAKINTLLPAQCTNGSEVRSSWAETTYWTSPQGNRETKIQDMDCVYYNWQYLVCSWKPGMGVH
FDTNYQLFYWYEGLDHSAECTDYIKVNGKNMGCRFPYLESSDYKDFYICVNGSSESQPIRPSYFIFQLQNI
VKPMPPDYLSLTVKNSEEINLKWNMPKGPIPAKCFIYEIEFTEDGTTWVTTTVENEIQITRTSNESQKLCE
LVRSKVNIYCSDDGIWSEWSDEQCWKGDIWKETPKRENGRVPRPPDCPKCPAPEMLGGPSVFIFPPKPKDT
LYITREPEVTCVVVDLDPEDPEVQISWFVDGKQMQTAKTQPREEQFNGTYRVVSVLPIGHQDWLKGKQFTC
KVNNKALPSPIERTISKARGQAHQPSVYVLPPSREELSKNTVSLTCLIKDFFPPDIDVEWQSNGQQEPESK
YRTTPPQLDEDGSYFLYSKLSVDKSRWQRGDTFICAVMHEALHNHYTQESLSHSPGK
cIL-4Rα-cIgGB-Fc-H
CH2 Numbering (N202H)
[SEQ ID NO: 11]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLPPKREN
GRVPRPPDCPKCPAPEMLGGPSVFIFPPKPKDTLLIARTPEVTCVVVDLDPEDPEVQISWFVDGKQMQTAK
TQPREEQFNGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWORGDTFICAVM
HEALHHHYTQESLSHSPGK
cIL-4Rα-cIgGB-Fc-YD
CH2 Numbering (L18Y/T22D)
[SEQ ID NO: 12]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLPPKREN
GRVPRPPDCPKCPAPEMLGGPSVFIFPPKPKDTLYIARDPEVTCVVVDLDPEDPEVQISWFVDGKQMQTAK
TQPREEQFNGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWORGDTFICAVM
HEALHNHYTQESLSHSPGK
CIL-13Rα2-cIgGB-Fc-YD
CH2 Numbering (L18Y/T22D)
[SEQ ID NO: 13]
SMLSNAEIKVNPPQDFEIVDPGYLGYLSLOWQPPLFPDNFKECTIEYELKYRNIDSENWKTIITKNLHYKD
GFDLNKGIEAKINTLLPAQCTNGSEVRSSWAETTYWTSPQGNRETKIQDMDCVYYNWQYLVCSWKPGMGVH
FDTNYQLFYWYEGLDHSAECTDYIKVNGKNMGCRFPYLESSDYKDFYICVNGSSESQPIRPSYFIFQLQNI
VKPMPPDYLSLTVKNSEEINLKWNMPKGPIPAKCFIYEIEFTEDGTTWVTTTVENEIQITRTSNESQKLCF
LVRSKVNIYCSDDGIWSEWSDEQCWKGDIWKETPKRENGRVPRPPDCPKCPAPEMLGGPSVFIFPPKPKDT
LYIARDPEVTCVVVDLDPEDPEVQISWFVDGKQMQTAKTQPREEQFNGTYRVVSVLPIGHQDWLKGKQFTC
KVNNKALPSPIERTISKARGQAHQPSVYVLPPSREELSKNTVSLTCLIKDFFPPDIDVEWQSNGQQEPESK
YRTTPPQLDEDGSYFLYSKLSVDKSRWQRGDTFICAVMHEALHNHYTQESLSHSPGK

The bold amino acid residues are the hinge regions, whereas the bold and underlined amino acid residues are substitutions to increase the in vivo half-life of the fusion proteins.

### Example 3

### Bispecific Fc Fusion Proteins

Bispecific cFc fusion proteins generally are considered a better alternative than homodimeric cFc fusion proteins because each of the two monomers of the bispecific cFc fusion proteins bind to a different target protein. In theory, this can substantially lower the overall manufacturing costs. Therefore, in one such bispecific cFc fusion protein generated comprised a heterodimer which consisted of the first monomer comprising in N-Terminal to C-Terminal order: the ECD of IL-13Rα1, the hinge region of IgGB, and the cFc of IgGB, whereas the second monomer in N-Terminal to C-Terminal order comprises the ECD of cIL-4Rα, the hinge region of IgGB, and the cFc of IgGB. Another bispecific cFc fusion protein comprised a heterodimer that consisted of a first monomer comprising in N-Terminal to C-Terminal order: the ECD of IL-13Rα2, the hinge region of IgGB, and the cFc of IgGB, whereas the second monomer in N-Terminal to C-Terminal order comprised the ECD of cIL-4Rα, the hinge region of IgGB, and the cFc of IgGB.

Heterodimeric proteins are synthesized and produced recombinantly from nucleotide sequences encoding the desired amino acid sequences similar to that described under Example 1 above. The heterodimeric proteins are administered to dogs via a variety of routes such as IV, SC, IP, or IM. Heterodimeric proteins may be administered at doses ranging from 0.1 ug/kg to 20 mg/kg or more. Typically, heterodimeric proteins may be administered at doses ranging from 0.1 mg/kg to 10 mg/kg.

In order to form bispecific fusion proteins, it is important to make amino acid substitutions on the Fc part of the fusion proteins of each of the binding partners in order to favor formation of a heterodimer over homodimer of the two Fc fusion proteins. Several potential ways or combinations of specific amino acid substitutions in the Fc part of the canine Fc fusion protein that may be used to favor heterodimer formation are provided in Table 1 below. These substitutions either favor a knobs-into-holes approach to heterodimerization of the Fc or favor an electrostatic attraction between different Fc chains to allow for heterodimerization [for a comprehensive discussion of these amino acid substitutions see, Moore et al., Methods, 154:38-50 (2019) and Brinkmann & Kontermann, MABS, 9:182-212 (2017)].

One bispecific fusion protein cIL-4Rα-13Rα1_ZW1-cFc, is a heterodimer of cIL-4Rα-cIgGB-Fc-ZW-A and cIL-13Rα1-cIgGB-Fc-ZW-B. Another bispecific fusion protein cIL-4Rα-13Rα2 ZW1-cFc is a heterodimer of cIL-4Rα-cIgGB-Fc-ZW-A and cIL-13Rα2-cIgGB-Fc-ZW-B.

TABLE 1
AMino Acid Replacements of CANINE IgG-B
Fc (SEQ ID NO: 2 or SEQ ID NO: 51)

| Number | Chain A | Chain B |
|---|---|---|
| (1) | T132W | T132S/L134A/Y175V |
| (2) | T132W/S120C | T132S/L134A/Y175V/Y115C |
| (3) | K177D/R160D | D167K/E122K |
| (4) | S130H/F173A | Y115T/T162F |
| (5) | F173L | K177R |
| (6) | L117Y/F173A/Y175V | T132L/R160L/T162W |
| (7) | K126E/K177W | S113R/D167V/F173T |
| (8) | K126E/K177W/Y115C | S113R/D167V/F173T/S120C |
| (9) | K136E/K177W | E123N/D167V/F173T |
| (10) | K126D/D167M/Y175A | Q111R/S113R/T132V/K177V |
| (11) | Y115S/K136Y/T132M/K177V | E122G/E123D/S130Q/Y175A |
| (12) | L117D/L134E | L117K/T132K |
| (13) | L134D/K136S | E122Q/S130K |

Examples of monomers of bispecific Fc fusion proteins of the present invention are listed below, and the amino acid replacements are in bold and underlined:

cIL-4Rα-cIgGB-Fc-ZW-A
[SEQ ID NO: 18]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLPPKREN
GRVPRPPDCPKCPAPEMLGGPSVFIFPPKPKDTLLIARTPEVTCVVVDLDPEDPEVQISWFVDGKQMQTAK
TQPREEQFNGTYRVVSVLPIGHQDWLKGKQFTCKVNNKALPSPIERTISKARGQAHQPSVYVYPPSREELS
KNTVSLTCLIKDFFPPDIDVEWQSNGQQEPESKYRTTPPQLDEDGSYALVSKLSVDKSRWQRGDTFICAVM
HEALHNHYTQESLSHSPGK
CIL-13Rαl-cIgGB-Fc-ZW-B
[SEQ ID NO: 19]
GGVAAPTETQPPVTNLSVSVENLCTVIWTWDPPEGASPNCTLRYFSHEDNKQDKKIAPETHRSKEVPLNER
ICLQVGSQCSTNESDNPSILVEKCTPPPEGDPESAVTELQCVWHNLSYMKCTWLPGRNTSPDTNYTLYYWH
SSLGKILQCEDIYREGQHIGCSFALTNLKDSSFEQHSVQIVVKDNAGKIRPSFNIVPLTSHVKPDPPHIKR
LFFQNGNLYVQWKNPQNFYSRCLSYQVEVNNSQTETNDIFYVEEAKCONSEFEGNLEGTICFMVPGVLPDT
LNTVRIRVRTNKLCYEDDKLWSNWSQAMSIGENTDPTPKRENGRVPRPPDCPKCPAPEMLGGPSVFIFPPK
PKDTLLIARTPEVTCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLLCLIKDFFPPDIDVEWQSNGQQE
PESKYLTWPPQLDEDGSYFLYSKLSVDKSRWQRGDTFICAVMHEALHNHYTQESLSHSPGK
CIL-13Rα2-cIgGB-Fc-ZW-B
[SEQ ID NO: 20]
SMLSNAEIKVNPPQDFEIVDPGYLGYLSLOWQPPLFPDNFKECTIEYELKYRNIDSENWKTIITKNLHYKD
GFDLNKGIEAKINTLLPAQCTNGSEVRSSWAETTYWTSPQGNRETKIQDMDCVYYNWQYLVCSWKPGMGVH
FDTNYQLFYWYEGLDHSAECTDYIKVNGKNMGCRFPYLESSDYKDFYICVNGSSESQPIRPSYFIFQLQNI
VKPMPPDYLSLTVKNSEEINLKWNMPKGPIPAKCFIYEIEFTEDGTTWVTTTVENEIQITRTSNESQKLCE
LVRSKVNIYCSDDGIWSEWSDEQCWKGDIWKETPKRENGRVPRPPDCPKCPAPEMLGGPSVFIFPPKPKDT
LLIARTPEVTCVVVDLDPEDPEVQISWFVDGKQMQTAKTQPREEQFNGTYRVVSVLPIGHQDWLKGKQFTC
KVNNKALPSPIERTISKARGQAHQPSVYVLPPSREELSKNTVSLLCLIKDFFPPDIDVEWQSNGQQEPESK
YLTWPPQLDEDGSYFLYSKLSVDKSRWQRGDTFICAVMHEALHNHYTQESLSHSPGK

Prior art amino acid sequences of the four canine IgGs:

cIgGA
Prior Art
[SEQ ID NO: 1]
LGGPSVLIFPPKPKDILRITRTPEVTCVVLDLGREDPEVQISWFVDGKEVHTAKTQSREQQENGTYRVVSV
LPIEHQDWLTGKEFKCRVNHIDLPSPIERTISKARGRAHKPSVYVLPPSPKELSSSDTVSITCLIKDFYPP
DIDVEWQSNGQQEPERKHRMTPPQLDEDGSYFLYSKLSVDKSRWQQGDPFTCAVMHETLQNHYTDLSLSHS
PGK
cIgGB
Prior Art
[SEQ ID NO: 2]
LGGPSVFIFPPKPKDTLLIARTPEVTCVVVDLDPEDPEVQISWFVDGKQMQTAKTQPREEQFNGTYRVVSV
LPIGHQDWLKGKQFTCKVNNKALPSPIERTISKARGQAHQPSVYVLPPSREELSKNTVSLTCLIKDFFPPD
IDVEWQSNGQQEPESKYRTTPPQLDEDGSYFLYSKLSVDKSRWQRGDTFICAVMHEALHNHYTQESLSHSP
GK
cIgGC
Prior Art
[SEQ ID NO: 3]
LGGPSVFIFPPKPKDILVTARTPTVTCVVVDLDPENPEVQISWFVDSKQVQTANTQPREEQSNGTYRVVSV
LPIGHQDWLSGKQFKCKVNNKALPSPIEEIISKTPGQAHQPNVYVLPPSRDEMSKNTVTLTCLVKDFFPPE
IDVEWQSNGQQEPESKYRMTPPQLDEDGSYFLYSKLSVDKSRWQRGDTFICAVMHEALHNHYTQISLSHSP
GK
cIgGD
Prior Art
[SEQ ID NO: 4]
LGGPSVFIFPPKPKDILRITRTPEITCVVLDLGREDPEVQISWFVDGKEVHTAKTQPREQQENSTYRVVSV
LPIEHQDWLTGKEFKCRVNHIGLPSPIERTISKARGQAHQPSVYVLPPSPKELSSSDTVTLTCLIKDFFPP
EIDVEWQSNGQPEPESKYHTTAPQLDEDGSYFLYSKLSVDKSRWQQGDTFTCAVMHEALQNHYTDLSLSHS
PGK

Prior art amino acid sequences of the canine IgG hinge regions for the four canine IgGs:

| | cIgGA hinge region |
|---|---|
| | Prior Art |
| [SEQ ID NO: 21] | |
| | FNECRCTDTPPCPVPEP |
| | cIgGB hinge region |
| | Prior Art |
| [SEQ ID NO: 22] | |
| | PKRENGRVPRPPDCPKCPT²PEM |
| | cIgGC hinge region |
| | Prior Art |
| [SEQ ID NO: 23] | |
| | AKECECKCNCNNCPCPGCGL |
| | cIgGD hinge region |
| | Prior Art |
| [SEQ ID NO: 24] | |
| | PKESTCKCISPCPVPES |

Prior art amino acid sequences of the ECDs of cIL-4Rα, cIL-13Rα1, and cIL-13Rα2

cIL-4Rα
Prior Art
[SEQ ID NO: 48]
VKVLHEPSCFSDYISTSVCQWKMDHPTNCSAELRLSYQLDEMGSENHTCVPENREDSVCVCSMPIDDAVEA
DVYQLDLWAGQQLLWSGSFQPSKHVKPRTPGNLTVHPNISHTWLLMWTNPYPTENHLHSELTYMVNVSNDN
DPEDFKVYNVTYMGPTLRLAASTLKSGASYSARVRAWAQTYNSTWSDWSPSTTWLNYYEPWEQHLP
cIL-13Rα1
Prior Art
[SEQ ID NO: 49]
GGVAAPTETQPPVTNLSVSVENLCTVIWTWDPPEGASPNCTLRYFSHEDNKQDKKIAPETHRSKEVPLNER
ICLQVGSQCSTNESDNPSILVEKCTPPPEGDPESAVTELQCVWHNLSYMKCTWLPGRNTSPDTNYTLYYWH
SSLGKILQCEDIYREGQHIGCSFALTNLKDSSFEQHSVQIVVKDNAGKIRPSFNIVPLTSHVKPDPPHIKR
LFFQNGNLYVQWKNPQNFYSRCLSYQVEVNNSQTETNDIFYVEEAKCONSEFEGNLEGTICFMVPGVLPDT
LNTVRIRVRTNKLCYEDDKLWSNWSQAMSIGENTDPT
cIL-13Rα2
Prior Art
[SEQ ID NO: 50]
SMLSNAEIKVNPPQDFEIVDPGYLGYLSLQWQPPLFPDNFKECTIEYELKYRNIDSENWKTIITKNLHYKD
GFDLNKGIEAKINTLLPAQCTNGSEVRSSWAETTYWTSPQGNRETKIQDMDCVYYNWQYLVCSWKPGMGVH
FDTNYQLFYWYEGLDHSAECTDYIKVNGKNMGCREPYLESSDYKDFYICVNGSSESQPIRPSYFIFQLQNI
VKPMPPDYLSLTVKNSEEINLKWNMPKGPIPAKCFIYEIEFTEDGTTWVTTTVENEIQITRTSNESQKLCF
LVRSKVNIYCSDDGIWSEWSDEQCWKGDIWKET

TABLE 2A
CANINE cFc's and HINGE REGIONS

| IgG cFc or Hinge Region | SEQ ID NO: |
|---|---|
| IgGA cFc(Prior Art) | 1 |
| IgGB cFc(Prior Art) | 2 |
| IgGC cFc (Prior Art) | 3 |
| IgGD cFc (Prior Art) | 4 |
| IgGA Hinge Region (Prior Art) | 21 |
| IgGB Hinge Region (Prior Art) | 22 |
| IgGC Hinge Region (Prior Art) | 23 |
| IgGD Hinge Region (Prior Art) | 24 |

TABLE 2B
HOMODIMERIC FUSION PROTEINS

| cFc Fusion protein | SEQ ID NO: |
|---|---|
| cIL-4Rα-cIgGB-Fc | 5 |
| cIL-13Rα1-cIgGB-Fc | 6 |
| cIL-13Rα2-cIgGB-Fc | 7 |
| cIL-4Rα-cIgGB-Fc-YD | 8 |
| cIL-13Rα1-cIgGB-Fc-YTE | 9 |
| cIL-13Rα2-cIgGB-Fc-YTE | 10 |
| cIL-4Rα-cIgGB-Fc-H | 11 |
| cIL-4Rα-cIgGB-Fc-YD | 12 |
| cIL-13Rα2-cIgGB-Fc-YD | 13 |

TABLE 2C
HETERODIMERIC FUSION PROTEINS SEQUENCES

| cFc Fusion protein | SEQ ID NO: |
|---|---|
| cIL-4Rα-cIgGB-Fc-ZW-A | 18 |
| cIL-13Rα1-cIgGB-Fc-ZW-B | 19 |
| cIL-13Rα2-cIgGB-Fc-ZW-B | 20 |

### Example 4

### Binding of the cFc FUSION PROTEINS TO CANINE IL-4 AND IL-13

### Methods:

The binding constants for the cFc fusion proteins provided in Tables 3 and 4 below, were determined using OCTET® HTX. All kinetics measurements were performed by OCTET® HTX using SA® biosensors and DATA ACQUISITION® 12.0 software. 10 μg/mL of biotin-labeled antigen, either canine IL-4 (cIL-4) or canine IL-13 (cIL-13) were loaded onto the SAR biosensors for 120 seconds. Next, the biosensors were placed into 1×pH 7.0 TBS/Casein buffer for 60 seconds for the blocking phase. For the association phase, antigen loaded biosensors were placed into 2-fold serial dilutions from 1 μM down to 15.6 nM of the wild-type, the bispecific, or the FcRn-mutant receptor Fc-fusions that recognized the cIL-4 or cIL-13 antigen in 1×pH 7.0 TBS/Casein buffer for 30 seconds. The last well was buffer alone and that sensor was used for reference sensor subtraction. Finally, the biosensors were placed into 1×pH 7.0 TBS/Casein buffer for 120 seconds for the dissociation phase. The results were then analyzed using Data Analysis 12.0 software and the curves were fitted using a 1:1 binding model.

### Results:

The association rate constant (ka), the dissociation rate constant (kdis), and the dissociation constant (KD) for the cIL-4Rα-cFc and the cIL-13Rα1-cFc, and cIL-13Rα2-cFc homodimeric and heterodimeric fusion proteins are provided in Tables 3 and 4 below. As can be seen from Table 3 below, the binding constant (KD) for the unmodified cIL-4Rα-cFc homodimer with cIL-4 was about 1×10⁻¹² M. In marked contrast, the KD for the heterodimeric bispecific cIL4Rα-IL13Rα1 ZW1-cFc with cIL-4 was about 10,000 times higher (about 1×10⁻⁸ M). In other words, the homodimeric cIL-4Rα-cFc binds about four orders of magnitude tighter to cIL-4 than the heterodimeric bispecific cIL4Rα-IL13Rα1_ZW1-cFc. Notably, the KD for the binding of the modified homodimer, cIL-4Rα-cFc-H to canine IL-4 (1×10⁻¹⁰) was approximately two orders of magnitude higher than that of the unmodified cIL-4Rα-Fc homodimer and the KD for the binding of the modified homodimer, cIL-4Rα-cFc-YTE to cIL-4 (1×10⁻¹¹) was approximately one order of magnitude higher than that of the modified homodimer. Moreover, the binding of the heterodimeric bispecific cIL4Rα-IL13Rα2_ZW1-cFc to cIL-4 was undetectable by this assay. Accordingly, modification of the homodimer cIL-4Rα-cFc to increase its half-life led to about 10 to 100-fold loss in affinity for cIL-4 for the homodimeric cIL-4Rα-cFc-NH and cIL-4Rα-cFc-YTE respectively, whereas the cIL4Rα-IL13Rα1 ZW1-cFc heterodimer showed a loss of about four orders of magnitude in affinity for cIL-4 relative to that of the unmodified cIL-4Rα-cFc homodimer, and the affinity of the dimeric cIL4Rα-IL13Rα2_ZW1-cFc for cIL-4 was so low it was undetectable under by this experimental procedure.

TABLE 3
IL-4 BINDING KINETICS

| cFc Fusion protein | KD (M) | ka (M⁻¹s⁻¹) | kdis (s⁻¹) |
|---|---|---|---|
| cIL-4Rα-cFc | 1.18E−12 | 7.03E+05 | 8.30E−07 |
| cIL-4Rα-cFc-YTE | 1.11E−11 | 8.30E+05 | 9.23E−06 |
| cIL-4Rα-cFc-H | 1.26E−10 | 7.85E+05 | 9.88E−05 |
| cIL4Rα-IL13Rα1_ZW1-cFc | 9.85E−09 | 2.37E+05 | 2.34E−03 |
| cIL4Rα-IL13Rα2_ZW1-cFc | ND | ND | ND |

ND = Binding Not Detected

The binding constant (KD) of the unmodified cIL-13Rα1-cFc with cIL-13 was about 5×10⁻⁹ M (see, Table 4 below). Modifying cIL-13Rα1-cFc to further increase its half-life, i.e., to make cIL-13Rα1-cFc-YTE, resulted in about a 4-fold decrease in the affinity for cIL-13, i.e., the KD increased to 2×10⁻⁸ M. Notably however, the heterodimeric bispecific cIL4Rα-IL13Rα1 ZW1-cFc exhibited an 8-fold tighter binding to cIL-13 (about 4×10⁻¹⁰ M; see, Table 4 below) than the unmodified IL-13Rα1-cFc. This is in direct contrast to the case with the exact same heterodimer binding to cIL-4, in which as pointed out above, the binding decreased by almost four orders of magnitude relative the corresponding cIL-4Rα-cFc homodimer (see, Table 3 above). These results indicate that forming a heterodimer can result in dramatic differences on the binding affinity of the two individual monomers of the heterodimer for their respective binding partners.

From Table 4 below, it is clear that the unmodified cIL-13Rα2-cFc binds extremely tightly to cIL-13, having a KD of about 7.5×10⁻¹³ M. Indeed, the binding of the cIL-13Rα2-cFc homodimer with cIL-13 is between three to four orders of magnitudes tighter than the binding of the cIL-13Rα1-cFc homodimer with cIL-13. Modifying cIL-13Rα2-cFc to either cIL-13Rα2-cFc-YTE or cIL-13Rα2-cFc-YD to increase its half-life resulted in a very modest increase in binding affinity for cIL-13, i.e., decreasing the KD by about a factor of two (to about 4.0×10⁻¹³ M). Strikingly, both homodimers of cIL-13Rα2-cFc-YTE or cIL-13Rα2-cFc-YD bind cIL-13 approximately four orders of magnitude tighter than the heterodimeric bispecific cIL-4Rα-IL13Rα2_ZW1-cFc, which has a KD of about 4×10⁻⁹ M (see, Table 4 below).

TABLE 4
IL-13 BINDING KINETICS

| cFc Fusion protein | KD (M) | ka (M⁻¹s⁻¹) | kdis (s⁻¹) |
|---|---|---|---|
| cIL-13Rα1-cFc | 5.37E−09 | 5.80E+04 | 3.12E−04 |
| cIL-13Rα1-cFc-YTE | 1.65E−08 | 6.65E+04 | 1.10E−03 |
| cIL4Rα-IL13Rα1_ZW1-cFc | 4.15E−10 | 9.44E+04 | 3.91E−05 |
| cIL-13Rα2-cFc | 7.44E−13 | 1.00E+06 | 7.45E−07 |
| cIL-13Rα2-cFc-YTE | 4.32E−13 | 1.17E+06 | 5.04E−07 |
| cIL-13Rα2-cFc-YD | 4.40E−13 | 1.15E+06 | 5.07E−07 |
| cIL-4Rα-IL13Rα2_ZW1-cFc | 4.15E−09 | 3.26E+04 | 1.35E−04 |

In summary, whereas the binding affinity for IL-4 significantly decreases when the homodimer of cIL-4Rα-cFc is replaced with a heterodimer of cIL-4Rα-cFc-ZW-A with either cIL-13Rα1-cFc-ZW-B or cIL-13Rα2-cFc-ZW-B, forming cIL4Rα-IL13Rα1_ZW1-cFc and cIL-4Rα-IL13Rα2_ZW1-cFc, respectively, the decrease in affinity is substantially greater for the cIL-4Rα-IL13Rα2 ZW1-cFc heterodimer. The corresponding binding affinity of IL-13, on the other hand, increases when the homodimer of cIL-13Rα1-cFc is replaced with the cIL4Rα-IL13Rα1 ZW1-cFc heterodimer, whereas the binding affinity for IL-13 substantially decreases when the homodimer of cIL-13Rα2-cFc is replaced by the cIL-4Rα-IL13Rα2 ZW1-cFc heterodimer.

### Example 5

### Inhibition of Stat-6 Phosphorylation by Cfc Fusion Proteins

The ability of the cFc fusion proteins to block the signaling mediated by IL-4 and IL-13, as measured by inhibition of STAT-6 phosphorylation in DH82 cells, were determined as follows:

### Materials:

1. Actively growing DH82 cells: Merck Animal Health Lot: 628-011, 24Oct14
2. HBSS, 1×: Corning, Catalog 21-022-CM
3. AlphaLISA p-STAT6 (Tyr641) Assay Kit: Perkin Elmer, Catalog ALSU-PST6-A10K
4. Recombinant canine IL-4: R&D Systems, Catalog: 752-CL/CF
5a. cFc fusion protein samples for IL-4 studies
   (i) cIL4Rα-cFc
   (ii) cIL4Rα-IL13Rα1 ZW1-cFc
   (iii) cIL4Rα-IL13Rα2 ZW1-cFc
5b. cFc fusion protein samples for IL-13 studies:
   (i) CIL13Rα1-cFc
   (ii) cIL13Rα2-cFc
   (iii) cIL4R-IL13Rα1 ZW1-cFc
   (iv) cIL4R-IL13Rα2 ZW1-cFc
6. Perkin Elmer® Envision

### Methods:

1. Tissue culture plates were seeded with 1×10⁵ DH82 cells per well (40 μL with the density of 2.5×10⁵ cells/mL) and incubated at 37° C. for 2 hours.
2. The cFc fusion proteins were pre-diluted to 2000 nM (500 nM final concentration in the well) and then 3-fold serially diluted in Hank's Balanced Salt Solution (HBSS). The proteins were added by transferring 20 μL/well to the respective locations on the tissue culture plates containing DH82 cells.
3. (a) Canine IL-4 was diluted to 10 ng/ml in HBSS (2.5 ng/ml in the well) and 20 μL was added to each well of the plates. The plates were incubated for 15 min at 37° C.; or alternatively
   (b) Canine IL-13 was diluted to 20 ng/ml in HBSS (5 ng/ml in the well) and 20 μL was added to each well of the plates. The plates were incubated for 15 min at 37° C.
4. The plates were removed from the incubator and 20 μL of 4× Lysis buffer from the AlphaLISA® p-STAT-6 Assay Kit was added to each well of the plate. The plate was agitated on a plate shaker with 350 rpm for 10 minutes at room temperature.
5. The Acceptor Mix was prepared from the AlphaLISA® p-STAT6 Assay Kit and 15 μL per well was added to 30 μL of the cell lysate in 96-well ½ Area Plates. The plates were sealed, agitated for 2 minutes at 350 rpm, and then incubated for 1 hour at room temperature.
6. The Donor Mix was prepared from the AlphaLISA® p-STAT6 Assay kit under subdued laboratory lighting and 15 μL per well was added to each plate. The plates were sealed, covered with foil, agitated for 2 minutes at 350 rpm, and then incubated for 1 hour at room temperature.
7. The plates were read using the AlphaScreen settings on the Perkin Elmer® En Vison.

### Results:

(a) The IC50 for inhibition of cIL-4 mediated STAT6 phosphorylation by cIL4Rα-cFc, cIL4Rα-IL13Rα1 ZW1-cFc, and cIL4Rα-IL13Rα2_ZW1-cFc in DH82 are provided in Table 5A below. As can be seen both the homodimeric cIL-4Rα-cFc and the heterodimeric bispecific cIL-4Rα-IL-13Rα1 ZW1-cFc inhibit 50% of the IL-4 mediated STAT6 phosphorylation at about a concentration of 80 pM and about 50 pM respectively, whereas the cIL-4Rα-IL-13Rα2 ZW1-cFc inhibits 50% of the cIL-4 mediated STAT6 phosphorylation at a concentration (i.e., about 0.2 μM) that is over 3 orders of magnitude higher than for either cIL4Rα-cFc or cIL4Rα-IL13Rα1 ZW1-cFc. Interestingly, in direct contrast with the heterodimeric cIL-4Rα-IL13R2α construct, the heterodimeric cIL-4Rα-IL13R1α construct binds at least as well, if not tighter to cIL-4 (see, Table 4) than the homodimeric cIL-4Rα-cFc and this relationship is consistent with the IC₅₀ data in Table 5A.

(b) The IC₅₀ for inhibition of cIL-13 mediated STAT6 phosphorylation by cIL13Rα1-cFc, cIL13Rα2-cFc, cIL4Rα-IL13Rα1_ZW1-cFc, and cIL4Rα-IL13Rα2 ZW1-cFc in DH82 cells are provided in Table 5B below. As can be seen, cIL-13Rα2-cFc inhibits 50% of the cIL-13 mediated STAT6 phosphorylation at about a concentration of 165 pM, whereas cIL13Rα1-cFc, cIL4Rα-IL13Rα1_ZW1-cFc, and cIL4Rα-IL13Rα2_ZW1-cFc all inhibit 50% of the cIL-13 mediated STAT6 phosphorylation well above nanomolar concentrations. Accordingly, though the concentration of cIL-4Rα-IL-13Rα1 ZW1-cFc to inhibit 50% of the cIL-13 mediated STAT6 phosphorylation is 6-fold lower than for cIL-13Rα1-cFc, it is still about 20-fold higher than for cIL-13Rα2-cFc, whereas the concentration of cIL-4Rα-IL-13Rα2_ZW1-cFc that inhibits 50% of the cIL-13 mediated STAT6 phosphorylation is about 200-fold higher than that for cIL-13Rα2-cFc. Therefore, the unmodified cIL-13Rα2-cFc homodimer surprisingly not only binds more tightly to cIL-13 than cIL-4Rα-IL-13Rα2_ZW1-cFc (see, Table 4 above), but it consistently also inhibits cIL-13 mediated STAT6 phosphorylation at substantially lower concentration than that found for cIL-4Rα-IL-13Rα2_ZW1-cFc (see, Table 5B below).

TABLE 5A
IL-4 IC₅₀

| cFc Fusion protein | IL-4 IC₅₀ (nM) |
|---|---|
| cIL-4Rα-cFc | 0.084 |
| cIL-4Rα-IL-13Rα1_ZW1-cFc | 0.046 |
| cIL-4Rα-IL-13Rα2_ZW1-cFc | 183.8 |

TABLE 5B
IL-13 IC₅₀

| cFc Fusion protein | IL-13 IC₅₀ (nM) |
|---|---|
| cIL-13Rα1-cFc | 18.44 |
| cIL-13Rα2-cFc | 0.165 |
| cIL-4Rα-IL-13Rα1_ZW1-cFc | 3.025 |
| cIL-4Rα-IL-13Rα2_ZW1-cFc | 29.45 |

Table 6 below summarizes the results obtained correlating the dissociation constants of the various binding partners with their respective IC₅₀'s. As can be seen, the optimal cytokine traps are homodimers of cIL-4Rα-cFc with homodimers of cIL-13Rα2-cFc.

TABLE 6
SUMMARY TABLE

| cFc Fusion protein | Form | IL-4 (KD) | IL-4 (IC₅₀) | IL-13 (KD) | IL-13 (IC₅₀) |
|---|---|---|---|---|---|
| cIL-4Rα-cFc | Homodimer | 1 × 10⁻¹² M | 8 × 10⁻¹¹ M | | |
| cIL-4Rα-IL-13Rα1 | Heterodimer | 1 × 10⁻⁸ M | 5 × 10⁻¹¹ M | 4 × 10⁻¹⁰ M | 3 × 10⁻⁹ M |
| cIL-4Rα-IL-13Rα2 | Heterodimer | ND | 2 × 10⁻⁷ M | 4 × 10⁻⁹ M | 3 × 10⁻⁸ M |
| cIL-13Rα1-cFc | Homodimer | | | 5 × 10⁻⁹ M | 2 × 10⁻⁸ M |
| cIL-13Rα2-cFc | Homodimer | | | 7 × 10⁻¹³ M | 2 × 10⁻¹⁰ M |

ND = Binding Not Detected

### Example 6

### Prior Art Caninized Antibodies to Canine IL-31

Antibodies that may be useful in the current invention are those described in U.S. Pat. No. 9,206,253B2 and U.S. Pat. No. 10,150,810B2. Preferably these antibodies have the following Light chain and Heavy chain sequences:

Caninized heavy chain sequence from mouse
antibody clone M14 and canine IgG-B:
Prior Art
[SEQ ID NO: 14]
EVQLVESGPSLVKPGGSLRLTCSVTGDSITSGYWNWIRKFPGNKLEYMGYISYSGITDYNPSLKSRITISR
DTSKNQYYLQLNSVTTEDTATYYCARYGNYGYAMDYWGQGTLVTVSSASTTAPSVEPLAPSCGSTSGSTVA
LACLVSGYFPEPVTVSWNSGSLTSGVHTFPSVLQSSGLYSLSSMVTVPSSRWPSETFTCNVAHPASKTKVD
KPVPKRENGRVPRPPDCPKCPAPEMLGGPSVFIFPPKPKDTLLIARTPEVTCVWDLDPEDPEVQISWFVDG
KQMQTAKTQPREEQFAGTYRWSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSVDKSRWQRGDT
FICAVMHEALHNHYTQESLSHSPGK
Caninized light chain sequence from mouse
antibody clone M14 and canine light chain
constant region:
Prior Art
[SEQ ID NO: 15]
DIVMTQSPASLSVSLGQRATISCRASESVDTYGNSEMHWYQQKPGQSPKLLIYRASNLESGIPAREGGSGS
GTDFTLTIDPVQADDVATYYCQQSYEDPWTFGGGTKLEIKRNDAQPAVYLFQPSPDQLHTGSASWCLLNSF
YPKDINVKWKVDGVIQDTGIQESVTEQDKDSTYSLSSTLTMSSTEYLSHELYSCEITHKSLPSTLIKSFQR
SECQRVD
Z-HC: Caninized heavy chain sequence:
Prior Art
[SEQ ID NO: 16]
EVQLVESGGDLVKPGGSLRLSCVASGFTFSNYGMSWVRQAPGKGLOWVATISYGGSYTYYPDNIKGRFTIS
RDNAKNTLYLQMNSLRAEDTAMYYCVRGYGYDTMDYWGQGTLVTVSSASTTAPSVFPLAPSCGSTSGSTVA
LACLVSGYFPEPVTVSWNSGSLTSGVHTFPSVLQSSGLYSLSSMVTVPSSRWPSETFTCNVAHPASKTKVD
KPVPKRENGRVPRPPDCPKCPAPEMLGGPSVFIFPPKPKDTLLIARTPEVTCVVVDLDPEDPEVQISWEVD
GKQMQTAKTQPREEQFNGTYRVVSVLPIGHQDWLKGKQFTCKVNNKALPSPIERTISKARGQAHQPSVYVL
PPSREELSKNTVSLTCLIKDFFPPDIDVEWQSNGQQEPESKYRTTPPQLDEDGSYFLYSKLSVDKSRWQRG
DTFICAVMHEALHNHYTQESLSHSPG
Z-LC: Caninized light chain sequence:
Prior Art
[SEQ ID NO: 17]
EIVMTQSPASLSLSQEEKVTITCKASQSVSFAGTGLMHWYQQKPGQAPKLLIYRASNLEAGVPSRESGSGS
GTDESFTISSLEPEDVAVYYCQQSREYPWTFGQGTKLEIKRNDAQPAVYLFQPSPDQLHTGSASVVCLLNS
FYPKDINVKWKVDGVIQDTGIQESVTEQDKDSTYSLSSTLTMSSTEYLSHELYSCEITHKSLPSTLIKSFQ
RSEC

TABLE 7
PRIOR ART IL-31 ANTIBODY SEQUENCES:

| | SEQ ID NO: | HC | LC |
|---|---|---|---|
| Caninized Mouse Antibody Clone | 14 | ✓ | |
| M14 and Canine IgG-B | | | |
| Caninized Mouse Antibody Clone | 15 | | ✓ |
| M14 and Canine LC Constant Region | | | |
| Z-HC: Caninized HC Sequence | 16 | ✓ | |
| Z-LC: Caninized LC Sequence | 17 | | ✓ |

### Example 7

### Caninized Antibodies to IL-31Rα

### Rat Monoclonal Antibodies Against Canine IL-31Rα:

Monoclonal antibodies against canine IL-31Rα were produced by the immunization of rats multiple times with the extracellular domain (ECD) of canine IL-31Rα (using 25 μg of antigen/anima each time) over a 3 to 4 weeks period. Following immunization, sera was collected from each animal and tested against canine IL-31Rα ECD by ELISA. The lymph node cells of the animals with the highest IL-31Rα ECD reactivity were fused with the myeloma SP2/0 cell line to produce hybridomas. Approximately 10 days after the fusion, supernatants from growing hybridomas were screened on IL-31Rα ECD protein coated plates by ELISA using the protocol described below. Three rat monoclonal antibodies were selected for caninization: 44E3, 10A12 and 28F12. These caninized antibodies bind tightly to canine IL-31Rα.

### The Procedure for the ELISA:

1. Coat 96-well half area plates with IL-31Rα (1 μg/mL in PBS buffer), 25 μL/well. Incubate the plates at 4° C. overnight.
2. Wash the plates 3 times with PBST (PBS +0.05% Tween 20)
3. Block the plates with blocking buffer (PBS with 5% FBS), 25 ul/well for 30 minutes at room temperature.
4. Transfer 25 ul/well hybridoma supernatant to the 96-well plates, incubate 60 minutes at room temperature.
5. Wash the plates 3 times by PBST.
6. Add 25 ul/well anti-rat IgG-HRP conjugate, 1:4000 dilution in blocking buffer, to the plates and incubate 60 minutes at room temperature.
7. Wash the plates 5 times by PBST.
8. Add TMB based reagent to the plates for colorimetric reaction for 20-30 minutes.
9. Stop the reactions with 0.16M sulfuric acid.
10. Read the plates by plate reader.

Using this procedures several hybridomas secreting antibodies that react with canine IL-31Rα were identified.

### Generation of Caninized Antibodies and Binding of Caninized Antibodies to Canine IL-31Rα:

The nucleotide and deduced amino acid sequence of the HC and LC of selected rat antibodies reactive with canine IL-31Rα was determined. The amino acid sequences representing the 3 HC CDRs and 3 LC CDRs for each antibody also were determined. These CDRs were used to develop caninized antibodies that bind canine IL-31Rα ECD. The binding of caninized antibodies to IL-31Rα was determined by ELISA as follows:

### Materials:

1. Anti-Dog IgG (cFc specific)-Peroxidase (Sigma-Aldrich SAB3700109-1.5 MG)
2. TMB-ELISA Substrate (Thermo-Fisher Cat #34028)
3. PBS pH7.4 (Thermo-Fisher Cat #10010001)
4. Tris Buffered Saline with Tween 20 (TBST) (Sigma-Aldrich T9039-10PAK)

### Method:

1. Coat immunoplates by cIL-31Rα in PBS buffer at 1 μg/mL, 100 μL/well. Incubate the plates at 37° C. for 1-2 hrs or 4° C. overnight.
2. Wash the plates 3 times by TBST buffer.
3. Block the plates by blocking buffer (0.5% BSA in TBST) for 45-60 minutes at room temperature.
4. Three-fold dilute anti-cIL31-Ra antibodies with blocking buffer in dilution plates and transfer the diluted antibodies into the cIL-31Rα coated plates, incubate 45-60 minutes at room temperature.
5. Wash the plates 3 times by TBST.
6. Add 1:2000 diluted HRP conjugated anti-dog IgG Fc into the plates, incubate 45-60 minutes at room temperature.
7. Wash the plates 3 times by TBST.
8. Add TMB-ELISA substrate into the plates for colorimetric reaction for 10 to 15 minutes.
9. Stop the reactions by 1 M H₃PO₄.
10. Read the plates by plate reader at 450 nM.

### Results:

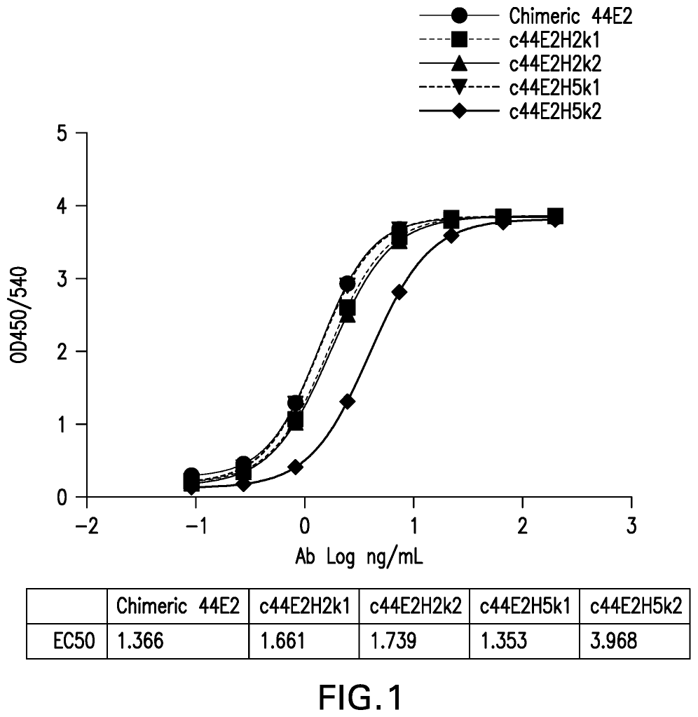

FIG. 1 shows the binding activity of related chimeric and caninized anti-canine IL-31Rα antibodies as evaluated by ELISA. Different designs of rat antibody 44E2 were assessed in the ELISA. The ELISA results indicate that though all of the caninized antibodies bind to canine IL-31Rα with an EC50 similar to the EC50 of the chimeric 44E2 antibody, c44E2 H5k1 binds to canine IL-31Rα with EC50 most similar to the EC50 of the corresponding chimeric 44E2 antibody.

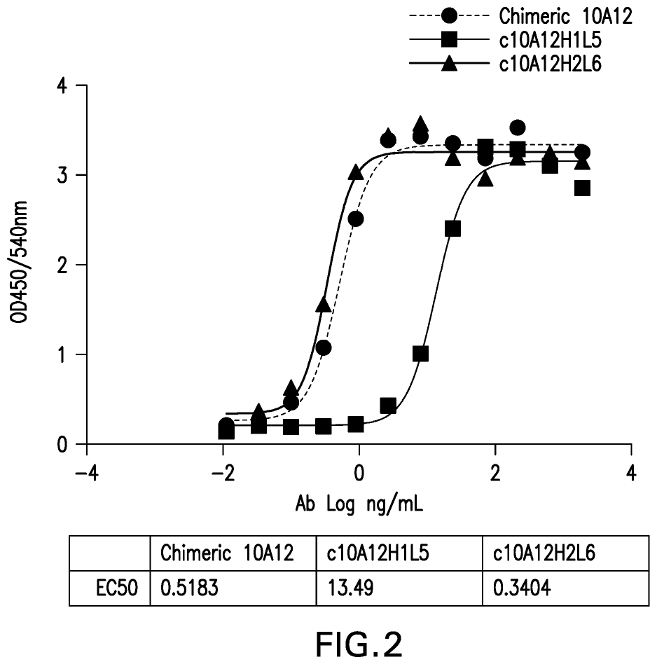

FIG. 2 shows the binding activity of related chimeric and caninized anti-canine IL-31Rα antibodies evaluated by ELISA. Different designs of rat antibody 10A12 were assessed in the ELISA. The ELISA results indicate that one of the caninized antibodies (c10A12 H2L6) binds to canine IL-31Rα with EC50 that is even lower than the EC50 for the corresponding chimeric 10A12 antibody.

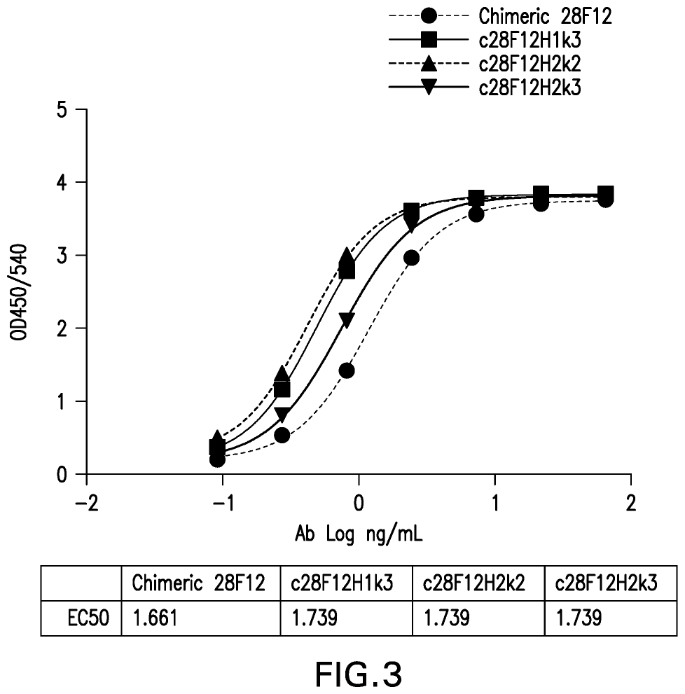

FIG. 3 depicts the binding activity of related chimeric and caninized anti-canine IL-31Rα antibodies evaluated by ELISA. Different designs of rat antibody 28F12 were assessed in the ELISA. The ELISA results indicate that the caninized antibodies bind to canine IL-31Rα with an even lower EC50 than the EC50 for the chimeric 28F12 antibody.

The following are examples of the amino acid sequences of chimeric (rat-canine) and caninized antibodies used in the present invention. The amino acids representing the CDRs are underlined.

r10A12VH-cIgGBm
[SEQ ID NO: 25]
EVQLVESGGGLVKPGRSMKLSCAASGFTFSNYYMAWVRQAPTKGLEWVASISTGGGNTYYRDSVKGRFTIS
RDNAKRTLYLQMDSLRSEDTATYYCGRHGTLYFDYWGQGVMVTVSSASTTAPSVEPLAPSCGSTSGSTVAL
ACLVSGYFPEPVTVSWNSGSLTSGVHTFPSVLOSSGLYSLSSMVTVPSSRWPSETFTCNVAHPASKTKVDK
PVPKRENGRVPRPPDCPKCPAPEMLGGPSVFIFPPKPKDTLLIARTPEVTCVVVALDPEDPEVQISWFVDG
KOMQTAKTQPREEQFAGTYRVVSVLPIGHQDWLKGKQFTCKVNNKALPSPIERTISKARGQAHQPSVYVLP
PSREELSKNTVSLTCLIKDFFPPDIDVEWQSNGQQEPESKYRTTPPQLDEDGSYFLYSKLSVDKSRWQRGD
TFICAVMHEALHNHYTQESLSHSPGK
c10A12VH1-cIgGBm
[SEQ ID NO: 26]
EVQLVESGGDLVKPGGSLRLSCVASGFTFSNYYMAWVRQAPGKGLQWVASISTGGGNTYYRDSVKGRFTIS
RDNAKNTLYLQMNSLRAEDTAMYYCAKHGTLYFDYWGQGTLVTVSSASTTAPSVEPLAPSCGSTSGSTVAL
ACLVSGYFPEPVTVSWNSGSLTSGVHTFPSVLQSSGLYSLSSMVTVPSSRWPSETFTCNVAHPASKTKVDK
PVPKRENGRVPRPPDCPKCPAPEMLGGPSVFIFPPKPKDTLLIARTPEVTCVVVALDPEDPEVQISWFVDG
KQMQTAKTQPREEQFAGTYRVVSVLPIGHQDWLKGKQFTCKVNNKALPSPIERTISKARGQAHQPSVYVLP
PSREELSKNTVSLTCLIKDFFPPDIDVEWQSNGQQEPESKYRTTPPQLDEDGSYFLYSKLSVDKSRWQRGD
TFICAVMHEALHNHYTQESLSHSPGK
c10A12VH2-cIgGBm
[SEQ ID NO: 27]
EVQLVESGGDLVKPGGSLRLSCAASGFTFSNYYMAWVRQAPGKGLQWVASISTGGGNTYYRDSVKGRFTIS
RDNAKNTLYLQMNSLRAEDTAMYYCARHGTLYFDYWGQGTLVTVSSASTTAPSVEPLAPSCGSTSGSTVAL
ACLVSGYFPEPVTVSWNSGSLTSGVHTFPSVLOSSGLYSLSSMVTVPSSRWPSETFTCNVAHPASKTKVDK
PVPKRENGRVPRPPDCPKCPAPEMLGGPSVFIFPPKPKDTLLIARTPEVTCVVVALDPEDPEVQISWFVDG
KQMQTAKTQPREEQFAGTYRVVSVLPIGHQDWLKGKQFTCKVNNKALPSPIERTISKARGQAHQPSVYVLP
PSREELSKNTVSLTCLIKDFFPPDIDVEWQSNGQQEPESKYRTTPPQLDEDGSYFLYSKLSVDKSRWQRGD
TFICAVMHEALHNHYTQESLSHSPGK
r10A12VL-cCl
[SEQ ID NO: 28]
QFTLTQPKSVSGSLRSTITIPCERSSGDIGDSYVSWYQQHLGRPPINVIYVDDQRPSEVSDRFSGSIDSSS
NSASLTITDLQMDDEADYFCQSYDSNIDGPVFGGGTKLTVLGQPKASPSVTLFPPSSEELGANKATLVCLI
SDFYPSGVTVAWKADGSPVTQGVETTKPSKQSNNKYAASSYLSLTPDKWKSHSSFSCLVTHEGSTVEKKVA
PAECS
c10A12VL4-cCl
[SEQ ID NO: 29]
QSVLTQPASVSGSLGQRVTISCERSSGDIGDSYVSWYQQLPGKAPSLLIYVDDQRPSGVPERFSGSKSGSS
NSATLTITGLQAEDEADYYCQSYDSNIDGPVFGGGTHLTVLGQPKASPSVTLFPPSSEELGANKATLVCLI
SDFYPSGVTVAWKADGSPVTQGVETTKPSKQSNNKYAASSYLSLTPDKWKSHSSFSCLVTHEGSTVEKKVA
PAECS
c10A12VL5-cCl
[SEQ ID NO: 30]
QPVLTQPPSLSASLGTTARLTCERSSGDIGDSYVSWYQQKPGSPPRDLLYVDDQRPSGVSKSFSGSKDTSA
NAGLLLISGLQPEDEADYYCQSYDSNIDGPVFGGGTHLTVLGQPKASPSVTLFPPSSEELGANKATLVCLI
SDFYPSGVTVAWKADGSPVTQGVETTKPSKQSNNKYAASSYLSLTPDKWKSHSSFSCLVTHEGSTVEKKVA
PAECS
c10A12VL6-cCl
[SEQ ID NO: 31]
QPVLTQPPSLSASLGTTARLTCERSSGDIGDSYVSWYQQKPGSPPRDVIYVDDQRPSEVSKSFSGSKDTSA
NAGLLLISGLQPEDEADYFCQSYDSNIDGPVEGGGTHLTVLGQPKASPSVTLFPPSSEELGANKATLVCLI
SDFYPSGVTVAWKADGSPVTQGVETTKPSKQSNNKYAASSYLSLTPDKWKSHSSFSCLVTHEGSTVEKKVA
PAECS
r28F12VH-cIgGBm
[SEQ ID NO: 32]
EVQLVESDGGLAQPGRSLKLSCAASGFTFSDYYMAWVRQAPTKGLEWVATISYDGSSTYYRDSVRGRFTIS
RDNAKSTLYLQMDSLRSEDTATYYCARGPLTDWAPNWFAYWGQGTLVTVSSASTTAPSVEPLAPSCGSTSG
STVALACLVSGYFPEPVTVSWNSGSLTSGVHTFPSVLQSSGLYSLSSMVTVPSSRWPSETFTCNVAHPASK
TKVDKPVPKRENGRVPRPPDCPKCPAPEMLGGPSVFIFPPKPKDTLLIARTPEVTCVVVALDPEDPEVQIS
WFVDGKQMQTAKTQPREEQFAGTYRVVSVLPIGHQDWLKGKQFTCKVNNKALPSPIERTISKARGQAHQPS
VYVLPPSREELSKNTVSLTCLIKDFFPPDIDVEWQSNGQQEPESKYRTTPPQLDEDGSYFLYSKLSVDKSR
WQRGDTFICAVMHEALHNHYTQESLSHSPGK
c28F12VH1-cIgGBm
[SEQ ID NO: 33]
EVQLVESGGDLVKPGGSLRLSCVASGFTFSDYYMAWVRQAPGKGLQWVATISYDGSSTYYRDSVRGRETIS
RDNAKNTLYLQMNSLRAEDTAMYYCAKGPLTDWAPNWFAYWGQGTLVTVSSASTTAPSVFPLAPSCGSTSG
STVALACLVSGYFPEPVTVSWNSGSLTSGVHTFPSVLQSSGLYSLSSMVTVPSSRWPSETFTCNVAHPASK
TKVDKPVPKRENGRVPRPPDCPKCPAPEMLGGPSVFIFPPKPKDTLLIARTPEVTCVVVALDPEDPEVQIS
WFVDGKQMQTAKTQPREEQFAGTYRVVSVLPIGHQDWLKGKQFTCKVNNKALPSPIERTISKARGQAHQPS
VYVLPPSREELSKNTVSLTCLIKDFFPPDIDVEWQSNGQQEPESKYRTTPPQLDEDGSYFLYSKLSVDKSR
WQRGDTFICAVMHEALHNHYTQESLSHSPGK
c28F12VH2-cIgGBm
[SEQ ID NO: 34]
EVQLVESGGDLVKPGGSLRLSCAASGFTFSDYYMAWVRQAPGKGLOWVATISYDGSSTYYRDSVRGRATIS
RDNAKNTLYLQMNSLRAEDTAMYYCARGPLTDWAPNWFAYWGQGTLVTVSSASTTAPSVEPLAPSCGSTSG
STVALACLVSGYFPEPVTVSWNSGSLTSGVHTFPSVLOSSGLYSLSSMVTVPSSRWPSETFTCNVAHPASK
TKVDKPVPKRENGRVPRPPDCPKCPAPEMLGGPSVFIFPPKPKDTLLIARTPEVTCVVVALDPEDPEVQIS
WFVDGKQMQTAKTQPREEQFAGTYRVVSVLPIGHQDWLKGKQFTCKVNNKALPSPIERTISKARGQAHQPS
VYVLPPSREELSKNTVSLTCLIKDFFPPDIDVEWQSNGQQEPESKYRTTPPQLDEDGSYFLYSKLSVDKSR
WQRGDTFICAVMHEALHNHYTQESLSHSPGK
r28F12VL-cCk
[SEQ ID NO: 35]
DIQMTQSPASLSASLGETVTIQCQTSEDIYSGLAWYQQKPGKSPQFLIYGASRLEDGVPSRFSGSGSGTQY
SLKISSMQTEDEGVYFCQQGLKYPNTFGAGTKLELKRNDAQPAVYLFQPSPDQLHTGSASVVCLLNSFYPK
DINVKWKVDGVIQDTGIQESVTEQDKDSTYSLSSTLTMSSTEYLSHELYSCEITHKSLPSTLIKSFQRSEC
QRVD
c28F12VL1-cCk
[SEQ ID NO: 36]
DIVMTQTPLSLSVSPGETASISCQTSEDIYSGLAWFRQKPGQSPQRLIYGASRLEDGVPDRFSGSGSGTDE
TLRISTVEADDTGVYYCQQGLKYPNTFGAGTKVELKRNDAQPAVYLFQPSPDQLHTGSASVVCLLNSFYPK
DINVKWKVDGVIQDTGIQESVTEQDKDSTYSLSSTLTMSSTEYLSHELYSCEITHKSLPSTLIKSFQRSEC
QRVD
c28F12VL2-cCk
[SEQ ID NO: 37]
EIVMTQSPASLSLSQEEKVTITCQTSEDIYSGLAWYQQKPGQAPKLLIYGASRLEDGVPSRFSGSGSGTDE
SFTISSLEPEDVAVYYCQQGLKYPNTFGAGTKVELKRNDAQPAVYLFQPSPDQLHTGSASVVCLLNSFYPK
DINVKWKVDGVIQDTGIQESVTEQDKDSTYSLSSTLTMSSTEYLSHELYSCEITHKSLPSTLIKSFQRSEC
QRVD
c28F12VL3-cCk
[SEQ ID NO: 38]
DIVMTQSPASLSLSQEEKVTITCQTSEDIYSGLAWYQQKPGQAPKLLIYGASRLEDGVPSRESGSGSGTDE
SFTISSLEPEDVAVYFCQQGLKYPNTFGAGTKVELKRNDAQPAVYLFQPSPDQLHTGSASVVCLINSFYPK
DINVKWKVDGVIQDTGIQESVTEQDKDSTYSLSSTLTMSSTEYLSHELYSCEITHKSLPSTLIKSFQRSEC
QRVD
c28F12VL4-cCk
[SEQ ID NO: 39]
DIVMTQTPLSLSVSPGETASISCQTSEDIYSGLAWFRQKPGQSPQLLIYGASRLEDGVPDRESGSGSGTDF
TLRISTVEADDTGVYFCQQGLKYPNTFGAGTKVELKRNDAQPAVYLFQPSPDQLHTGSASVVCLLNSFYPK
DINVKWKVDGVIQDTGIQESVTEQDKDSTYSLSSTLTMSSTEYLSHELYSCEITHKSLPSTLIKSFQRSEC
QRVD
r44E2VH-cIgGBm
[SEQ ID NO: 40]
QVQLKESGPGLVQPSQTLSLTCTVSGFSLTSNGVSWVRQPPGKGLEWIAAISSGGSTYYNSVLKSRLSISR
DTSKSQVELKMNSLQTEDTAIYFCTRRLSGYNYVPFAYWGQGTLVTVSSASTTAPSVEPLAPSCGSTSGST
VALACLVSGYFPEPVTVSWNSGSLTSGVHTFPSVLOSSGLYSLSSMVTVPSSRWPSETFTCNVAHPASKTK
VDKPVPKRENGRVPRPPDCPKCPAPEMLGGPSVFIFPPKPKDTLLIARTPEVTCVVVALDPEDPEVQISWF
VDGKQMQTAKTQPREEQFAGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKSRWQ
RGDTFICAVMHEALHNHYTQESLSHSPGK
c44E2VH1-cIgGBm
[SEQ ID NO: 41]
EVQLVESGGDLVKPEGSLRLSCVVSGFTFSSNGVSWVRQAPGKGLQWVAAISSGGSTYYNSVLKSRFTISR
DNAKNTLYLQMNSLRTEDTAVYYCAKRLSGYNYVPFAYWGQGTLVTVSSASTTAPSVFPLAPSCGSTSGST
VALACLVSGYFPEPVTVSWNSGSLTSGVHTFPSVLOSSGLYSLSSMVTVPSSRWPSETFTCNVAHPASKTK
VDKPVPKRENGRVPRPPDCPKCPAPEMLGGPSVFIFPPKPKDTLLIARTPEVTCVVVALDPEDPEVQISWE
VDGKQMQTAKTQPREEQFAGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKSRWQ
RGDTFICAVMHEALHNHYTQESLSHSPGK
c44E2VH4-cIgGBm
[SEQ ID NO: 42]
ELTLQESGPGLVKPSQTLSLTCVVSGGSVTSNGVSWIRQRPGRGLEWMGAISSGGSTYYNSVLKSRISITA
DTAKNQFSLQLSSMTTEDTAVYYCARRLSGYNYVPFAYWGQGTLVTVSSASTTAPSVEPLAPSCGSTSGST
VALACLVSGYFPEPVTVSWNSGSLTSGVHTFPSVLQSSGLYSLSSMVTVPSSRWPSETFTCNVAHPASKTK
VDKPVPKRENGRVPRPPDCPKCPAPEMLGGPSVFIFPPKPKDTLLIARTPEVTCVVVALDPEDPEVQISWF
VDGKQMQTAKTQPREEQFAGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKSRWQ
RGDTFICAVMHEALHNHYTQESLSHSPGK
c44E2VH5-cIgGBm
[SEQ ID NO: 43]
ELTLQESGPGLVKPSQTLSLTCTVSGFSLTSNGVSWIRQRPGRGLEWMGAISSGGSTYYNSVLKSRISITA
DTAKNQFSLQLSSMTTEDTAVYYCARRLSGYNYVPFAYWGQGTLVTVSSASTTAPSVEPLAPSCGSTSGST
VALACLVSGYFPEPVTVSWNSGSLTSGVHTFPSVLQSSGLYSLSSMVTVPSSRWPSETFTCNVAHPASKTK
VDKPVPKRENGRVPRPPDCPKCPAPEMLGGPSVFIFPPKPKDTLLIARTPEVTCVVVALDPEDPEVQISWF
VDGKQMQTAKTQPREEQFAGTYRVVSVLPIGHQDWLKGKQFTCKVNNKALPSPIERTISKARGQAHQPSVY
VLPPSREELSKNTVSLTCLIKDFFPPDIDVEWQSNGQQEPESKYRTTPPQLDEDGSYFLYSKLSVDKSRWQ
RGDTFICAVMHEALHNHYTQESLSHSPGK
r44E2VL-cCk
[SEQ ID NO: 44]
DIQMTQSPSLLSASVGDRVTLNCKASQNIYKHLAWCQQKLGEPPNLLISNANSLQTGIPSRESGSGSGTDE
TLTISSLOPEDVATYFCQQYYSGDTFGAGTKLELKRNDAQPAVYLFQPSPDQLHTGSASVVCLLNSFYPKD
INVKWKVDGVIQDTGIQESVTEQDKDSTYSLSSTLTMSSTEYLSHELYSCEITHKSLPSTLIKSFQRSECQ
RVD
c44E2VL1-cCk
[SEQ ID NO: 45]
EIVMTQSPASLSLSQEEKVTITCKASQNIYKHLAWYQQKPGQAPKLLIYNANSLQTGVPSRFSGSGSGTDE
SFTISSLEPEDVAVYYCQQYYSGDTFGAGTKVELKRNDAQPAVYLFQPSPDQLHTGSASVVCLLNSFYPKD
INVKWKVDGVIQDTGIQESVTEQDKDSTYSLSSTLTMSSTEYLSHELYSCEITHKSLPSTLIKSFQRSECQ
RVD
c44E2VL2-cCk
[SEQ ID NO: 46]
EIVMTQSPASLSLSQEEKVTITCKASQNIYKHLAWYQQKPGQAPKLLIYNANSLQTGIPSRESGSGSGTDE
SFTISSLEPEDVAVYFCQQYYSGDTFGAGTKVELKRNDAQPAVYLFQPSPDQLHTGSASVVCLLNSFYPKD
INVKWKVDGVIQDTGIQESVTEQDKDSTYSLSSTLTMSSTEYLSHELYSCEITHKSLPSTLIKSFQRSECQ
RVD
c44E2VL4-cCk
[SEQ ID NO: 47]
EIVMTQSPGSLAGSAGESVSINCKASQNIYKHLAWYQQKPGERPKLLIYNANSLQTGVPARFSSSGSGTDE
TLTINNLQAEDVGDYYCQQYYSGDTFGAGTKVELKRNDAQPAVYLFQPSPDQLHTGSASVVCLLNSFYPKD
INVKWKVDGVIQDTGIQESVTEQDKDSTYSLSSTLTMSSTEYLSHELYSCEITHKSLPSTLIKSFQRSECQ
RVD

TABLE 8
IL-31R ANTIBODY SEQUENCES

| | SEQ ID | | |
|---|---|---|---|
| | NO: | HC | LC |
| r10A12VH-cIgGBm | 25 | ✓ | |
| c10A12VH1-cIgGBm | 26 | ✓ | |
| c10A12VH2-cIgGBm | 27 | ✓ | |
| r10A12VL-cCl | 28 | | ✓ |
| c10A12VL4-cCl | 29 | | ✓ |
| c10A12VL5-cCl | 30 | | ✓ |
| c10A12VL6-cCl | 31 | | ✓ |
| r28F12VH-cIgGBm | 32 | ✓ | |
| c28F12VH1-cIgGBm | 33 | ✓ | |
| c28F12VH2-cIgGBm | 34 | ✓ | |
| r28F12VL-cCk | 35 | | ✓ |
| c28F12VL1-cCk | 36 | | ✓ |
| c28F12VL2-cCk | 37 | | ✓ |
| c28F12VL3-cCk | 38 | | ✓ |
| c28F12VL4-cCk | 39 | | ✓ |
| r44E2VH-cIgGBm | 40 | ✓ | |
| c44E2VH1-cIgGBm | 41 | ✓ | |
| c44E2VH4-cIgGBm | 42 | ✓ | |
| c44E2VH5-cIgGBm | 43 | ✓ | |
| r44E2VL-cCk | 44 | | ✓ |
| c44E2VL1-cCk | 45 | | ✓ |
| c44E2VL2-cCk | 46 | | ✓ |
| c44E2VL4-cCk | 47 | | ✓ |

TABLE 9
PRIOR ART SEQUENCES OF ECDS OF THE
IL-13 AND IL-4 RECEPTOR α PROTEINS

| ECD | SEQ ID NO: |
|---|---|
| cIL-4Rα | 48 |
| cIL-13Rα1 | 49 |
| cIL-13Rα2 | 50 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A composition comprising a homodimer that comprises a pair of canine Interleukin-4 receptor alpha-canine fragment crystallizable region fusion proteins (cIL-4Rα-cFc fusion proteins) and a homodimer comprising a pair of canine Interleukin-13 receptor alpha 2-canine fragment crystallizable region fusion proteins (cIL-13Rα2-cFc fusion proteins);
wherein each one of said pair of cIL-4Rα-cFc fusion proteins comprises an extracellular domain (ECD) of canine Interleukin-4 receptor alpha (cIL-4Rα) or fragment thereof that binds canine Interleukin-4 (cIL-4), and a first canine fragment crystallizable region (cFc); and
wherein each one of said pair of cIL-13Rα2-cFc fusion proteins comprises an extracellular domain (ECD) of canine Interleukin-13 receptor alpha 2 (cIL-13Rα2) or fragment thereof that binds canine Interleukin-13 (cIL-13), and a second cFc;
wherein the first cFc and the second cFc are either the same or different.

2. The composition of claim 1, wherein the first cFc and the second cFc individually comprise an amino acid sequence that has at least 90%, 95%, 99%, or 100% identity with the amino acid sequence selected from the group consisting of SEQ ID NO: 1, SEQ ID NO: 2, SEQ ID NO: 3, SEQ ID NO: 4, or SEQ ID NO: 51.

3. The composition of claim 1, wherein each one of said pair of cIL-4Rα-cFc fusion proteins further comprises a first canine hinge region; wherein said first canine hinge region acts as a linker between the ECD of the cIL-4Rα and the first cFc; and
wherein each one of said pair of cIL-13Rα2-cFc fusion proteins further comprises a second canine hinge region; wherein said second canine hinge region acts as a linker between the ECD of the cIL-13Rα2 and the second cFc; wherein the first canine hinge region and the second canine hinge region are either the same or different.

4. The composition of claim 3, wherein the first canine hinge region and the second canine hinge region individually comprise an amino acid sequence that has at least 85%, 90%, 95%, or 100% identity with the amino acid sequence selected from the group consisting of SEQ ID NO: 21, SEQ ID NO: 22, SEQ ID NO: 23, and SEQ ID NO: 24.

5. The composition of claim 1, wherein the ECD of cIL-4Rα comprises at least 85%, 90%, 95%, or 100% identity with the amino acid sequence of SEQ ID NO: 48.

6. The composition of claim 1, wherein the ECD of cIL-13Rα2 comprises at least 85%, 90%, 95%, or 100% identity with the amino acid sequence of SEQ ID NO: 50.

7. The composition of claim 3, wherein said first canine hinge region acts as the sole linker between the ECD of the cIL-4Rα and the first cFc; and wherein the sole linker between the ECD of the cIL-4Rα and the first cFc comprises an amino acid sequence that is identical to an amino acid sequence in a protein naturally found in canines, including naturally occurring variants thereof.

8. The composition of claim 3, wherein said second canine hinge region acts as the sole linker between the ECD of the cIL-13Rα2 and the second cFc; and wherein the sole linker between the ECD of the cIL-13Rα2 and the second cFc comprises an amino acid sequence in a protein naturally found in canines, including naturally occurring variants thereof.

9. The composition of claim 1, wherein each one of said pair of cIL-4Rα-cFc fusion proteins is composed solely of amino acid sequences that are identical to amino acid sequences of proteins naturally found in canines, including naturally occurring variants thereof.

10. The composition of claim 1, wherein each one of said pair of cIL-13Rα2-cFc fusion proteins is composed solely of amino acid sequences that are identical to amino acid sequences of proteins naturally found in canines, including naturally occurring variants thereof.

11. The composition of claim 1, wherein each one of said pair of cIL-4Rα-cFc fusion proteins comprises an amino acid sequence that has at least 90%, 95%, or 99% identity with the amino acid sequence of SEQ ID NO: 5.

12. The composition of claim 11, wherein each one of said pair of cIL-4Rα-cFc fusion proteins comprises an amino acid sequence selected from the group consisting of SEQ ID NO: 5, SEQ ID NO: 8, SEQ ID NO: 11, and SEQ ID NO: 12.

13. The composition of claim 1, wherein each one of said pair of cIL-13Rα2-cFc fusion proteins comprises an amino acid sequence that has at least 90%, 95%, or 99% identity with the amino acid sequence of SEQ ID NO: 7.

14. The composition of claim 13, wherein each one of said pair of cIL-13Rα2-cFc fusion proteins comprises an amino acid sequence selected from the group consisting of SEQ ID NO: 7, SEQ ID NO: 10, and SEQ ID NO: 13.

15. The composition of claim 1, further comprising a canine antipruritic antibody or a caninized antipruritic antibody.

16. The composition of claim 15, wherein the canine antipruritic antibody or the caninized antipruritic antibody is selected from the group consisting of a caninized antibody against canine Interleukin-31 (cIL-31), a canine antibody against cIL-31, a caninized antibody against canine Interleukin-31R (cIL-31R), and a canine antibody against cIL-31R.

17. The composition of claim 16, wherein the caninized antibody against cIL-31 comprises:
(i) a heavy chain comprising the amino acid sequence of SEQ ID NO: 14 and a light chain comprising the amino acid sequence of SEQ ID NO: 15, or
(ii) a heavy chain comprising the amino acid sequence of SEQ ID NO: 16 and a light chain comprising the amino acid sequence of SEQ ID NO: 17.

18. The composition of claim 16, wherein the caninized antibody against cIL-31R is selected from the group consisting of:
(i) a heavy chain comprising the amino acid sequence selected from the group consisting of SEQ ID NO: 26 and SEQ ID NO: 27, and a light chain comprising the amino acid sequence selected from the group consisting of SEQ ID NO: 29, SEQ ID NO: 30, and SEQ ID NO: 31;
(ii) a heavy chain comprising the amino acid sequence selected from the group consisting of SEQ ID NO: 33 and SEQ ID NO: 34, and a light chain comprising the amino acid sequence selected from the group consisting of SEQ ID NO: 36, SEQ ID NO: 37, SEQ ID NO: 38 and SEQ ID NO: 39; and
(iii) a heavy chain comprising the amino acid sequence selected from the group consisting of SEQ ID NO: 41, SEQ ID NO: 42, and SEQ ID NO: 43, and a light chain comprising the amino acid sequence selected from the group consisting of SEQ ID NO: 45, SEQ ID NO: 46, and SEQ ID NO: 47.

19. The composition of claim 1, that further comprises one or more additional components selected from the group consisting of a Janus kinase (JAK) inhibitor, a spleen tyrosine kinase (SYK) inhibitor, or an antagonist to a chemoattractant receptor-homologous molecule expressed on TH2 cells.

20. The composition of claim 19, wherein the JAK inhibitor is selected from the group consisting of 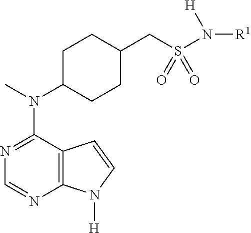
where R¹ is C₁₋₄ alkyl optionally substituted with hydroxy, and pharmaceutically acceptable salts thereof, 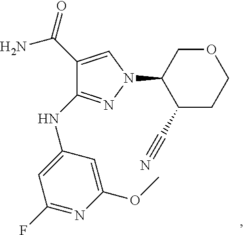 [SMILES: COc1cc(Nc2nn([C@H]3COCC[C@@H]3C#N)cc2C(N)=O)cc(F)n1]
and pharmaceutically acceptable salts thereof, and 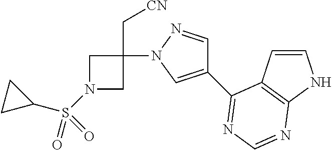 [SMILES: N#CCC1(n2cc(-c3ncnc4[nH]ccc34)cn2)CN(S(=O)(=O)C2CC2)C1]
and pharmaceutically acceptable salts thereof.

21. A method of treating atopic dermatitis comprising administering the composition of claim 1, to a canine that has atopic dermatitis.
